(19) United States
(12) Patent Application Publication
(10) Pub. No.: US 2007/0224273 A1
(43) Pub. Date: Sep. 27, 2007

(54) Multifunctional Supramolecular Hydrogels as Biomaterials

(76) Inventors: Bing Xu, Clear Water Bay (HK); Zhimou Yang, Clear Water Bay (HK); Gaolin Liang, Clear Water Bay (HK); Qigang Wang, Clear Water Bay (HK)

Correspondence Address: LAW OFFICES OF ALBERT WAI-KIT CHAN, LLC, WORLD PLAZA, SUITE 604, 141-07 20TH AVENUE, WHITESTONE, NY, 11357, US

(21) Appl. No.: 11/692,857

(22) Filed: Mar. 28, 2007

Related U.S. Application Data
(63) Continuation-in-part of application No. 11/237,498, filed on Sep. 27, 2005.
(63) Continuation-in-part of application No. PCT/US05/35112, filed on Sep. 27, 2005.
(60) Provisional application No. 60/613,413, filed on Sep. 28, 2004.
(60) Provisional application No. 60/613,413, filed on Sep. 28, 2004.
(60) Provisional application No. 60/878,053, filed on Jan. 3, 2007.

(51) Int. Cl. B01J 13/00; A61K 9/26; C12N 1/04; C12N 9/00; C12Q 1/25

(52) U.S. Cl. 424/488; 424/484; 435/183; 435/243; 435/4; 516/9

(57) ABSTRACT

The present invention provides supramolecular hydrogels having a three-dimensional, self-assembling, elastic, network structure comprising non-polymeric, functional molecules and a liquid medium, whereby the functional molecules are noncovalently crosslinked. The functional molecules may be, for instance, anti-inflammatory molecules, antibiotics, metal chelators, anticancer agents, small peptides, surface-modified nanoparticles, or a combination thereof. Applications of the present invention include use of the supramolecular hydrogel, for instance, as a biomaterial for wound healing, tissue engineering, drug delivery, and drug/inhibitor screening.

### CROSS-REFERENCE TO RELATED APPLICATIONS

This is a continuation-in-part application of U.S. Ser. No. 11/237,498, filed Sep. 27, 2005, which claims the benefit of U.S. Ser. No. 60/613,413, filed Sep. 28, 2004, now abandoned. This is also a continuation-in-part application of International Application No. PCT/US05/035112, filed Sep. 27, 2005, which claims the benefit of U.S. Ser. No. 60/613,413, filed Sep. 28, 2004, now abandoned. This application also claims the benefit of priority of U.S. Ser. No. 60/878,053, filed Jan. 3, 2007. The entire contents and disclosures of the preceding applications are incorporated by reference into this application.

Throughout this application, various references or publications are cited. Disclosures of these references or publications in their entireties are hereby incorporated by reference into this application in order to more fully describe the state of the art to which this invention pertains.

### FIELD OF THE INVENTION

This invention provides a three-dimensional, self-assembling supramolecular hydrogel comprising non-polymeric, functional molecules.

### BACKGROUND OF THE INVENTION

Hydrogels, formed by three-dimensional, elastic networks whose interstitial spaces are filled with a liquid, possess many useful properties (e.g., response to external stimuli, flow in response to shear force, etc.). Because of their useful properties, hydrogels have applications in many areas, such as bioanalysis, chemical sensing, food processing, cosmetics, drug delivery, and tissue engineering.

Following the successful applications of polymer-based hydrogels in biomedical engineering and the successful studies on low molecular weight organogels, supramolecular hydrogels formed by the self-assembly of small molecules have recently emerged as a new type of biomaterial that promises important biomedical applications (e.g., hydrogels based on the self-assembly of oligopeptides have been used as scaffolds to grow neurons). These oligopeptide-based hydrogels, however, are only mono-functional, and their cost remains high.

In contrast, the present invention pertains to a new type of supramolecular hydrogel, wherein the self-assembled nanofibers or nano-networks of functional small molecules (or entities) serve as the matrix to encapsulate water and to form the hydrogel. Additionally, these small molecules maintain their therapeutic effects even though they serve as the structural components of the supramolecular hydrogels. Because of their resemblance to the extracellular matrix, their biocompatibility, and their biodegradability, this type of hydrogel may serve as a new and general platform for diverse applications in biomedical areas, such as removal of toxics, wound healing, tissue engineering, and drug delivery.

### SUMMARY OF THE INVENTION

The present invention pertains to the general design and application of a new supramolecular hydrogel, whose self-assembled networks comprise one or more types of functional molecules (e.g., anti-inflammatory molecules, antibiotics, metal chelators, anticancer agents, small peptides, and/or surface-modified nanoparticles), as biomaterials for a range of applications, such as wound healing, tissue engineering, drug delivery, anticancer therapy, treatment of infectious diseases, drug/inhibitor screening, and removal of toxins.

The design of the supramolecular hydrogel includes: 1) modifying functional molecules to convert them into hydrogelators while enhancing or maintaining their therapeutic activities and 2) triggering the hydrogelation process by physical, chemical, or enzymatic processes, thereby resulting in the creation of a supramolecular hydrogel via formation of non-covalent crosslinks by the functional molecules. Notably, the functional molecules maintain their therapeutic effects even though they serve as the structural components of the supramolecular hydrogels.

In one embodiment, the present invention provides a supramolecular hydrogel having a three-dimensional, self-assembling, network structure comprising non-polymeric, functional molecules and a liquid medium, wherein the functional molecules are noncovalently crosslinked.

The present invention also provides a method of using the supramolecular hydrogel described herein to treat wounds.

The present invention also provides a method of making a supramolecular hydrogel comprising the use of a precursor of hydrogelators that are hydrolyzed by a hydrolyase under proper conditions, thereby generating hydrogelators that form the hydrogel.

The present invention also provides a method of screening an enzyme inhibitor based on such inhibitor's ability to inhibit the formation of hydrogel described herein.

The present invention also provides a method of screening for the presence of an enzyme based on such enzyme's ability to generate the hydrogel described herein.

The present invention also provides a method of using the hydrogel described herein to deliver a therapeutic agent.

The present invention also provides a method of conducting an enzymatic reaction by enclosing an active site of an enzyme in the hydrogel described herein.

The present invention also provides a method of using the hydrogel described herein as a three-dimensional matrix for cell culture.

### BRIEF DESCRIPTION OF THE DRAWINGS

FIG. 1 shows the structures of three small molecules: N-(Fluorenyl-9-methoxycarbonyl)-L-Leucine, N-(Fluorenyl-9-methoxycarbonyl)-L-Lysine, and pamidronate. N-(Fluorenyl-9-methoxycarbonyl)-L-Leucine 1 and N-(Fluorenyl-9-methoxycarbonyl)-L-Lysine 2 belong to a novel class of anti-inflammatory agents reported by Burch, et al. (1991), and 1 displays effective anti-inflammatory activity in animal models. Neither 1 nor 2 acts as a hydrogelator in a neutral aqueous solution. The addition of pamidronate (3) to the suspension of 1 and 2 leads to the formation of a hydrogel at pH=9, in which 3 acts as both a donor and an acceptor of hydrogen bonds to promote hydrogelation. In addition, 3 is a clinically-used drug and forms a stable complex with UO₂²⁺ and reduces the poison caused by the uranyl ions.

FIG. 2A shows weight change of the mice. Initial weights are normalized as 1; 0 represents deceased mice. Data are mean±SD obtained in N mice in the group, in which N=7, 7, 5 concerning the (−), (+), and healing groups, respectively. FIG. 2B shows a plausible interaction between the hydrogel and the simulated uranium wound.

FIG. 3A shows the molecular structures of the ligand, vancomycin 4, and the derivatives of the receptors 5, 6, and 7. FIG. 3B shows the linear viscoelastic frequency sweep responses of the hydrogels of 5 and 5+4 at strain of 1% and 0.1%, respectively. FIG. 3C shows the linear viscoelastic frequency sweep responses of the hydrogels of 6, 7, 6+4, and 7+4 at 1% strain. The concentrations of 4, 5, 6, and 7 are all 30 mM.

FIG. 4A and FIG. 4B show the structure of 8 and the optical image of the hydrogel of 8 (0.36 wt %) (taken by a flatbed scanner when the vial was laid horizontally).

FIG. 5 shows the molecular structures of two compounds used for the formation of hydrogels and the schematic gelation process. Conditions of gelation: (i) Na₂CO₃, buffer; (ii) enzyme, 37° C.; (iii) Na₂CO₃, buffer; and (iv) enzyme, 60° C. (buffer: pH˜9.6, 50 mM of Tris-HCl plus 1 mM of MgCl₂).

FIG. 6 shows an illustration of the design for identifying inhibitors of an enzyme by hydrogelation.

FIG. 7 shows results of activities of three inhibitors: row 1) Left to right: sol. of 9; sol. of 9 and enzyme; sol. of 9+ pamidronate; sol. of 9+ Zn²⁺; and sol. of 9+ Na3VO4 ([pamidronate]=[Zn2+]=[Na3VO4]=33 mM); row 2) pamidronate; row 3) Zn2+; and row 4) Na3VO4. (Left to right, Conc.=33; 3.3; 0.33; 0.033; 0.0033 mM).

FIG. 8A shows the solution of the hydrogelator at low concentration. FIG. 8B shows the formation of hydrogels after adding surface-modified magnetic nanoparticles, abbreviated as “NP”. FIG. 8C shows the effects of applying magnetic field, represented as “H”, to the hydrogel for 1 hour. FIG. 8D shows the effects of applying magnetic field, H, to the hydrogel for 4 hours. FIG. 8E shows the effects of applying magnetic field, H, to the hydrogel for 10 hours.

FIG. 9 shows the chemical structures of the naphthalene-containing dipeptide derivatives as the biocompatible hydrogelators.

FIG. 10 shows the frequency dependence of the storage moduli (G′: filled symbols) and the loss moduli (G″: open symbols) of hydrogels at the strain of 0.15% with concentrations at 0.5% of different hydrogels: ▪, 13; ●, 12; ▴, 11; and ▾, 14.

FIG. 11 shows the TEM images of hydrogels formed by compound 11 (FIG. 11A), compound 12 (FIG. 11B), compound 13 (FIG. 11C), and compound 14 (FIG. 11D) with the concentration at 0.5 wt %.

FIG. 12 shows the chemical structures of the pentapeptide derivatives 15, 16, 17, 18, 19, and 20. R=a, b, or c.

FIG. 13 shows the chemical structures of the β-amino acid derivatives 21 and 22.

FIG. 14 shows the optical images of the hydrogels of 21 (FIG. 14A) and 22 (FIG. 14B).

FIG. 15 shows the gelation properties of the pentapeptides 15a-c (SEQ. ID No. 1), 16a-c (SEQ. ID No. 2), 17a-c (SEQ. ID No. 3), 18a-c (SEQ. ID No. 4), 19a-c (SEQ. ID No. 5), and 20a-c (SEQ. ID No. 6).

FIG. 16 shows the design of a substrate of β-lactamase (Bla) as the precursor of a hydrogelator (X=S or COO); the opening of β-lactam ring by Bla; and one possible mode of the self-assembly of the hydrogelator and the formation of the hydrogel.

FIG. 17 shows the synthesis of a substrate of β-lactamase as a precursor of hydrogelator.

FIG. 18 shows the optical images and transmission electron microscopy (TEM) images of viscous solution of precursor 3 (A, C) and gel I (B, D).

FIG. 19 shows the chemical structures of the hydrogelators, Nap-L-Phe-D-Glucosamine (1) and Nap-D-Phe-D-Glucosamine (2).

FIG. 20 shows the optical images of Gel I (FIG. 20A) and Gel II (FIG. 20B); strain (FIG. 20C) and frequency dependence (FIG. 20D) of dynamic storage moduli (G′) and loss moduli (G″) of the hydrogels; TEM images of Gel I (FIG. 20E) and Gel II (FIG. 20F); circular dichroism (CD) of the hydrogels (FIG. 20G), and emission spectra (FIG. 20H) of 1 and 2 in solution and in the hydrogels.

FIG. 21 shows the gross appearance (FIGS. 21A, 21B), histological cross-section images (FIGS. 21C, 21D) and enlarged images (FIGS. 21E, 21F) of the dorsal skins of Balb/C mice on day 6 after wounding. FIGS. 21A, C, and E are negative control and FIGS. 21B, D, and F are Gel II-treated mice immediately after the incision was made. Histological specimens were embedded in paraffin wax and stained with hematoxylin and eosin. a, scar tissue; b, extracellular matrix (ECM); c, keratinocytes.

FIG. 22 shows the chemical structures of the hydrogelators, Nap-L-Phe-L-Phe (1), Nap-D-Phe-D-Phe (2), Nap-s-β³-HPhg-s-β³-HPhg (3) and Nap-L-fPhe-L-fPhe (4).

FIG. 23 shows the optical images of molecular hydrogels 6 hours after releasing folic acid: Gel I (FIG. 23A); Gel II (FIG. 23B); Gel III (FIG. 23C); and Gel IV (FIG. 23D). FIG. 23E shows digestion curve of four molecular hydrogelators upon treatment of proteinase K (the conversion determined by HPLC). FIG. 23F shows release curve of Folic acid from four kinds of gels, and FIG. 23G shows controlled release of Folic acid from Gel IV by proteinase K.

FIG. 24A shows the process of using enzyme to control the balance of hydrophilic and hydrophobic interactions to form a supramolecular hydrogel in vivo. FIG. 24B shows the chemical structures of the molecules for hydrogelation and their enzymatic conversions.

FIG. 25 shows the optical images of the hydrogels formed by 6.91 mM of compound 3 at pH=1.5 (FIG. 25A); with 10 μL of acid phosphatase at pH=4.8, 25° C., and concentrations of 5.88 U/ml (FIG. 25B), 2.94 U/ml (FIG. 25C), and 1.47 U/ml (FIG. 25D). FIG. 25E shows dynamic frequency sweep of Gel I at the strain of 1.0%; and FIGS. 25F-G show dynamic time sweep of the solution containing 0.5 wt % (6.91 mM) of 3 and 10 μL of acid phosphatase at concentration of 5.88 U/mL (FIG. 25F), 2.94 U/mL (FIG. 25G), and 1.47 U/mL (FIG. 25H), at the strain of 1.0% and the frequency of 2.0 rad/s. All rheological measurements were carried out at room temperature, pH=4.8.

FIG. 26 shows transmission electron micrographs of Gel I (FIG. 26A), Gel II (FIG. 26B), Gel III (FIG. 26C), and Gel IV (FIG. 26D).

FIG. 27 shows the optical images of hydrogelation in blood by mixing 0.3 mL of blood (from rabbit), 0.2 mL of solution of 3 (1.0 wt % in PBS buffer, pH=7.4), and 10 μL of alkali phosphatase (FIG. 27A); 0.3 mL of blood and 0.2 mL of solution of 3 (1.0 wt % in PBS buffer, pH=7.4) (FIG. 27B); 0.3 mL of blood, 0.2 mL of PBS buffer, and 10 μL of alkali phosphatase (FIG. 27C); and the gel formed by mixing 0.2 mL of solution of 3 (1.0 wt % in PBS buffer, pH=7.4), 10 μL of alkali phosphatase, and 1.0×10⁶ broken Hela cells (FIG. 27D).

FIG. 28 shows the optical images of injection sites of the mice (indicated by the arrows) immediately after administration of 0.5 mL of PBS buffer solution containing 3 (0.8 wt %) or 5 (0.8 wt %) and alkali phosphatase (5 μL, 50-150 U) (FIG. 28A); one hour after injections (FIG. 28B); two hours after injections (left: with solution 5 and enzyme; right: with solution of 3 and enzyme) (FIG. 28C); and a typical hydrogel formed at the injection site of a mouse (FIG. 28D).

FIG. 29A shows the structures of the molecules and the procedure for making the supramolecular hydrogels containing hemin chloride. FIG. 29B shows the artificial enzyme-catalyzed peroxidation of pyrogallol to purpurogallin (S: the substrate, P: the product, and Solvents: aqueous buffer (0.01 M, pH 7.4, phosphate) or toluene).

FIG. 30 shows the TEM images of Gel I (FIG. 30A) and Gel II (FIG. 30B); the AFM image of nanofiber in Gel II (FIG. 30C); the high resolution TEM image of Gel II (FIG. 30D), and the EDX analysis (FIGS. 30E and 30F) in the selected area in D.

FIG. 31 shows the UV-visible spectra of free hemin in pH 7.4 buffer (Hemin_{(Free)}), hemin in hydrogel (Hemin_{(Phe)}), and hemin in hydrogel with L-histidine (Hemin_{(Phe+His)}). The spectra of their weak absorption region were enlarged four times.

FIG. 32 shows the initial reaction courses of pyrogallol (10.0 mM) and H₂O₂ (40.0 mM) in 0.01 M pH 7.4 buffer by 5 μM Hemin_{(Phe+His)} (●), Hemin_{(Phe)} (▴), Hemin_{(Free)} (▪), and Gel I control with 0.5 mL/L concentration (▾). The reactions in the first minute displayed zero order kinetics, thus being defined as the initial activity.

FIG. 33 shows the 15 minutes reaction courses of pyrogallol (10.0 mM) and H₂O₂ (40.0 mM) catalyzed by Hemin_{(Phe+His)} in toluene (●), Hemin_{(Phe)} in toluene (▴), Hemin_{(Free)} in toluene (▪), Hemin_{(Phe+His)} in water (∘), Hemin_{(Phe)} in water (Δ), and Hemin_{(Free)} in water (□).

FIG. 34 shows the molecular hydrogel-immobilized enzymes that catalyze a reaction in organic media (light blue: water; yellow: organic phase; E: enzymes; S: substrates; P: products).

FIG. 35a shows a comparison of activities of Hb(I) and Hb(U) in various media. FIG. 35b shows the first minute reaction course of pyrogallol (10 mM) and H₂O₂ (30 mM) catalyzed by various Hb (0.1 g/L) displays zero order kinetics (all r>0.99), and is therefore being used to calculate the initial rate. FIG. 35e shows the ratios of activities of E(I) (I: immobilized by the molecular hydrogel) in toluene and E(U) (U: unconfined) in water. The observed activities of E(U) in water are labelled above the green bars. FIG. 35d shows the extended 15 minutes reaction course for the reactions shown in FIG. 35b. All the concentrations were calibrated according to the molar extinction coefficient of the product in different solvents.

### DETAILED DESCRIPTION OF THE INVENTION

Hydrogels are hydrophilic polymers that absorb water and are insoluble in water at physiologic temperature, pH, and ionic strength because of the presence of a three-dimensional network. Hydrogels may be present as interpenetrating polymer networks (IPNs) and block copolymers.

The area of hydrogel research has expanded dramatically in the last 10 years, primarily because hydrogels, both the synthetic and natural, perform well for biomedical applications. Hydrogels work well in the body because they mimic the natural structure of the body's cellular makeup. Recent advances in the use of hydrogels for tissue engineering, drug delivery, and other biomedical applications of hydrogels have led to the potential to design artificial organs in a controlled fashion and to deliver drugs to specific sites in the body.

Uses of Hydrogels

Drug Delivery. The goal of drug delivery is to maintain the drug concentration in the body (plasma) within therapeutic limits for long periods of time. Conventional drug administration (oral delivery, injection) usually results in poor control of the plasma drug concentration. The controlled release of drugs from polymeric matrices has, however, been very successful.

Tissue Engineering. Accidents and diseases lead to devastating tissue losses and organ failures, which result in more than 8 million surgical operations each year in the United States alone. These problems convert to a national annual healthcare cost of approximately half trillion U.S. dollars. The state-of-the-art clinical therapies to tissue losses and organ failures can be categorized into three approaches, i.e., transplantation, surgical reconstruction, and the use of prostheses. Although each of these approaches has contributed to solving or alleviating the severity of these clinical problems, all of them have serious limitations.

The goal of tissue engineering is to create living, three-dimensional tissue/organs using cells obtained from readily available sources. Amongst different approaches in tissue engineering, growing cells in 3-D matrices (scaffolds) or devices has become increasingly active. Hydrogels have been popular in certain tissue engineering applications because of the ability to fill irregularly shaped tissue defects and the ease of incorporation of cells or bioactive agents. The use of biodegradable hydrogels as a temporary-support template for cartilage (Ashiku et al., 1997) and for bone regeneration through growth factor release has been reported (Tabata et al., 1998).

Wound Dressings. Hydrogels have also been used as wound dressings because most hydrogels are soft, flexible, conform to the wound, are biocompatible, and are permeable to water vapor and metabolites. As wound dressings, they absorb the exudate, do not stick to the wound, allow for access of oxygen to the wound site, and accelerate healing.

Biosensors. A biosensor is a compact device or probe that detects, records, and transmits information regarding a physiological change or the presence of various chemical or biological materials in the environment. A biosensor is a probe that integrates a biological component e.g. enzyme or antibody, with an electronic component to yield a measurable response that are proportional to analyte(s). Biosensors are used to monitor changes in the physiological environment.

Hydrogels have been used as reactive matrix membranes in biosensors. Hydrogels possess several advantages over other materials in that they exhibit rapid and selective diffusion characteristics of the analyte, as well as provide support. Among the various types of biosensors, those that measure glucose have received the most attention. In these biosensors, the consumption of oxygen or the formation of hydrogen peroxide is monitored (enzyme glucose oxidase catalyzes the reaction of glucose and oxygen to form gluconic acid and hydrogen peroxide). Hydrogels are used as enzyme immobilization matrices in these types of biosensors.

As used herein, the term “hydrogel” refers to materials having water and three dimensional networks with or without additional other components.

As used herein, the term “supramolecular hydrogel” refers to the hydrogel whose three dimensional networks are formed by driving forces that are non-covalent interactions.

The term “non-polymeric” means that the molecules do not have covalently-linked repeating units. However, this invention does not exclude the use of polymers in combination with non-polymers.

As used herein, the term “small molecules” or “non-polymeric molecules” shall generally refer to molecules without covalently linked repeating units with certain exceptions. After reading the whole disclosure of this invention, one of ordinary skills in the art would appreciate that small peptides with repeating units such as Nap-D-Phe-D-Phe could be within the scope of this invention. Representative examples of non-polymeric molecules include, but are limited to, small peptides such as derivatives of single amino acids, dipeptides, tripeptides, β-amino acids, tetrapeptides and pentapetides, wherein the molecular weight of these derivatives are less than 3.0 kD. As used in the present disclosure, “small molecules” may be used interchangeably with “non-polymeric molecules”.

As used herein, the term “hydrogelator” refers to molecules that are the building block of the three dimensional networks in supramolecular hydrogels.

The present invention pertains to the design and application of a new type of supramolecular hydrogel having a three-dimensional, self-assembling, elastic, network structure comprising non-polymeric, functional molecules and a liquid medium, whereby said functional molecules are noncovalently crosslinked. The functional molecules (or entities) may be, for instance, anti-inflammatory molecules, antibiotics, metal chelators, anticancer agents, small peptides, surface-modified nanoparticles, or a combination thereof In general, the noncovalent crosslinking of the functional molecules is effectuated ligand-receptor interaction, hydrogen bonding, hydrophobic interaction, or ionic interaction.

In one embodiment, the antibiotics may be, for instance, vancomycin, penicillin, amoxicillin, cephalosporin, oxacillin, nafcillin, clindamycin, erythromycin, ciprofloxacin, rifampin, amphotericin, and/or sulfamethoxaole. The metal chelators may be chelating agents for radioactive isotopes, such as uranium chelating agents, cesium chelating agents, iodine chelating agents, stronium chelating agents, and/or americium chelating agents.

In another embodiment, examples of small peptides include single amino acids, dipeptides, tripeptides, tetrapeptides, β-amino acids, pentapetides, and derivatives thereof, wherein the molecular weight of the small peptides and derivatives are less than 3.0 kD.

In yet another embodiment, the non-polymeric Functional molecules comprise a naphthalene group.

In another embodiment, the hydrogels described herein comprise β-amino acids (e.g., β³-alanine, β³-phenylalaine, and β³-HPhg). Hydrogels comprising β-amino acids have enhanced biostability compared to hydrogels that do not contain β-amino acids.

In one embodiment, the liquid medium is retained within the interstitial spaces of the hydrogel structure. The liquid medium includes, but is not limited to, water, physiological saline, or other liquid medium. Examples of suitable liquid mediums have been identified so as to facilitate subsequent uses of the hydrogel.

The design of the supramolecular hydrogel includes: (1) modifying functional molecules to convert them into hydrogelators while enhancing or maintaining their therapeutic properties and (2) triggering the hydrogelation process, thereby resulting in the creation of a supramolecular hydrogel via formation of non-covalent crosslinks by the functional molecules.

The modification of step (1) includes attaching or removing one or more groups in the functional molecule. In step (2), the hydrogelation process may be triggered by physical, chemical, or enzymatic processes.

The present invention further provides a supramolecular hydrogel made by the above method.

The present invention also provides a method of using the supramolecular hydrogel described herein to treat wounds. In one embodiment, such hydrogel comprises non-polymeric functional molecules having a naphthalene group. In another embodiment, the non-polymeric functional molecules comprise naphthalene group and glucosamine.

The present invention also provides a method of making a supramolecular hydrogel, comprising the use of a precursor of hydrogelators that are hydrolyzed by a hydrolyase under proper conditions, thereby generating hydrogelators that form the hydrogel. Examples of hydrolyases include, but are not limited to, alkaline phosphatase and esterase, peptidases, amidases. Examples of hydrogelators include naphthalene or FMoc, phenyalanine, tyrosine phosphate, etc.

The present invention also provides a method of screening an enzyme inhibitor, comprising the steps of: providing a precursor which transforms into a hydrogelator in the presence of an enzyme; contacting the precursor with the enzyme; and determining the formation of hydrogel by the hydrogelator, wherein inhibition of hydrogel formation in the presence of the candidate compound indicates the candidate compound is an enzyme inhibitors. In general, the enzymes can be derived from bacteria, viruses, or parasites.

The present invention also provides a method of screening a test sample for the presence of an enzyme, comprising the steps of: providing a precursor which transforms into a hydrogelator in the presence of the enzyme; contacting the precursor with the test sample; and determining the formation of hydrogel by the hydrogelator, wherein hydrogel formation in the presence of the test sample indicates the test sample contains the enzyme. In general, the enzymes can be derived from bacteria, viruses, or parasites.

The present invention also provides a method of using the hydrogel described herein to deliver a therapeutic agent. In one embodiment, the hydrogel comprises β-amino acids.

The present invention also provides a method of conducting an enzymatic reaction by enclosing an active site of an enzyme in the hydrogel described herein. The enzymatic reaction can take place in water or organic solvent.

The present invention also provides a method of using the hydrogel described herein as a three-dimensional matrix for cell culture.

The invention being generally described, will be more readily understood by reference to the following examples which are included merely for purposes of illustration of certain aspects and embodiments of the present invention, and are not intended to limit the invention.

### EXAMPLE 1

### Wound Healing

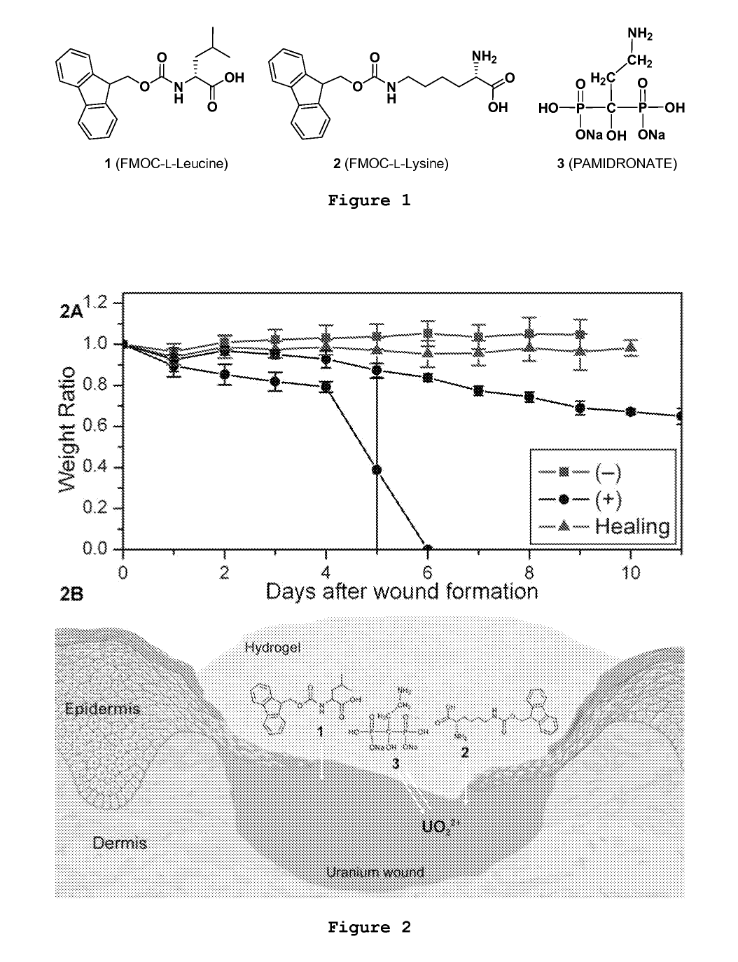

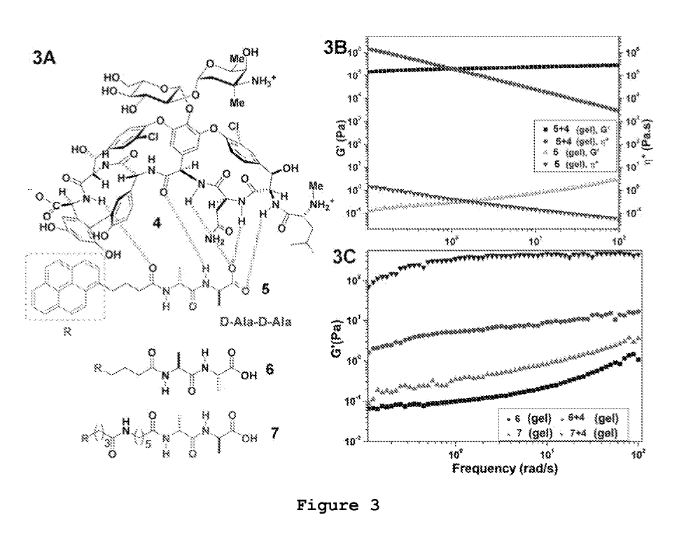

To illustrate the biological activity of the supramolecular hydrogel of the present invention, hydrogel comprising the functional molecules shown in FIG. 1 was used to treat a uranium wound, which was created by scratching the skin on the back of mice and externally administering uranyl nitrate to the wound. The hydrogel was then topically administered to the wounds of the negative control group 20 minutes afterwards but not for the positive control group. The results of the experiment are shown in FIG. 2A. The mice in all groups exhibited initial weight loss the next day due to the effects of the wound. The negative control group recovered quickly from the wound after experiencing slight initial weight-loss and returned to normal growth on day 2. In contrast, the positive control group showed continuous weight-loss until expiration in about five days or 35% weight-loss over the next ten days. Thus, when the hydrogel was administered topically to the uranyl nitrate wounds of the mice in the negative control group, the mice experienced little weight loss and a nice recovery, with none of the toxic effects of the uranyl nitrate being observed in the mice's daily behavior.

FIG. 2B depicts a plausible delivery process of the functional molecules shown in FIG. 1. Both 1 and 2 migrate into the wound to reduce the inflammation by blocking the recruitment of neutropils into the inflamed site, and 3 decreases the toxicity of UO₂²⁺ by chelating with UO₂²⁺. In addition, since the hydrogel is able to “uptake” UO₂²⁺ from a uranyl nitrate solution, the hydrogel absorbs some of the UO₂²⁺ from the wound site and, thus, further reduces the damage caused by UO₂²⁺.

Although the effectiveness against a wound caused by other radioactive elements remains to be tested, the present hydrogel can be used advantageously in the confinement of radioactive uranium compared to liquid-based treatments since the hydrogel absorbs UO22+ well and has little fluidity. Thus, the hydrogel of the present invention is usefull as an emergency treatment for uranium wounds. Accordingly, the above example demonstrates that other combinations of hydrogelators, selected from a pool of pharmaceutical molecules, may be used to create other useful biomaterials.

### EXAMPLE 2

### Noncovalent Crosslinking of Supramolecular Hydrogels

Although in-situ polymerization allows enhanced stability of small-molecular gels, such a covalent cross-linking approach usually requires additional chemical synthesis, which alters the properties of the hydrogelators, and may result in the loss of biocompatibility and biodegradability. Accordingly, the use of molecular recognition (noncovalent crosslinking) to enhance the elasticity of the small-molecular hydrogels is preferred. For instance, the addition of a ligand into the mechanically-weak hydrogels of a derivative of the receptor leads to up to a million-fold increase in the storage modulus of the hydrogel. The term “noncovalent crosslinking” means that the crosslinking is realized by hydrogen bonding, hydrophobic forces, or ionic forces.

In one embodiment, vancomycin (Van) was selected as the ligand 4 and a D-Ala-D-Ala derivative was selected as the receptor 5 because of the well-established molecular recognition (FIG. 3A) between 4 and 5 in aqueous solution. Compound 5 gels water at the minimum gelation concentration of ˜30 mM and pH 9.5. In contrast, the mixture of 4 and 5 (mole ratio=1:1) forms a hydrogel at the minimum gel concentration of 5 mM and pH=9.5. This type of ligand-receptor pairs can be used for constructing supramolecular hydrogels.

Dynamic oscillatory measurements were used to evaluate the viscoelastic behavior of these two hydrogels at the same concentration (30 mM). To ensure that the hydrogels are reversible upon applying a shear force, all the frequency sweep measurements followed the determination of the linear viscoelastic regime by a strain sweep. As shown in the linear viscoelastic frequency sweep response of the hydrogels (FIG. 3B), the storage modulus (G′) of the hydrogel of 5 is 0.12 Pa at 0.1 rad/s. The frequency dependence versus complex viscosity (η*∝(frequency)ⁿ⁻¹, n=0.47±0.006) and a nonlinear frequency response started at 100 rad/s indicate that 5 can form only a liquid-like hydrogel. At the concentration of 30 mM, G′ of the hydrogel of 5+4 is 1.6×10⁵ Pa at 0.1 rad/s, and its frequency dependence versus complex viscosity (η*∝(frequency)ⁿ⁻¹, n=0.1±0.006) indicates the solid-like and highly elastic features of the hydrogel. Increasing the molar ratio of 4 (compared to 5) from zero to one increases G′ of the hydrogel of 5+4, following a power law (G′∝[4]ₙ, n=5.93±0.31), suggesting that 4 acts as a crosslinker.

### EXAMPLE 3

### Antibiotic Supramolecular Hydrogels

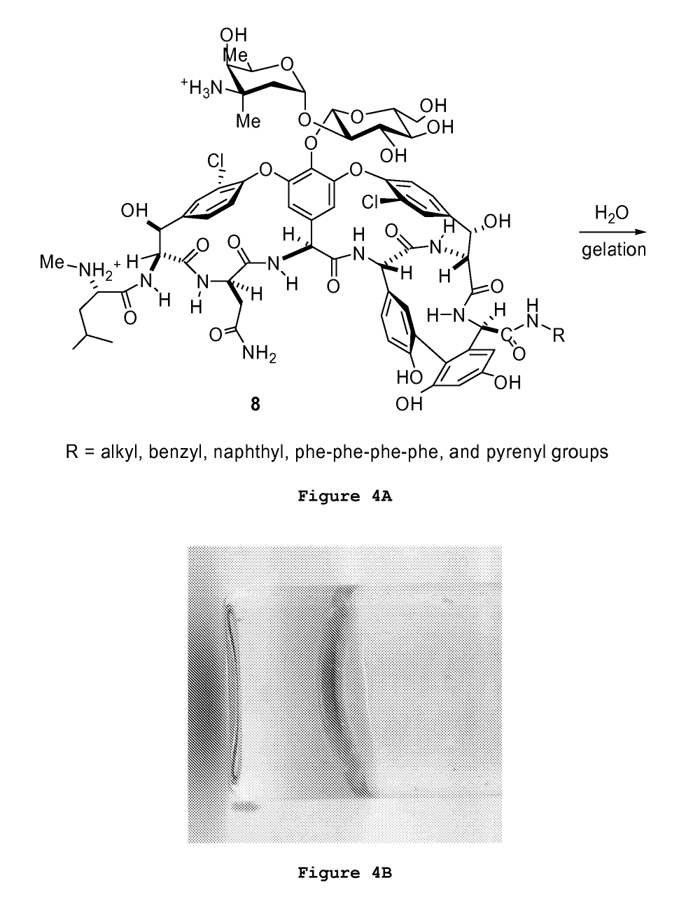

FIG. 4A shows the chemical structure of 8 (when R=pyrenyl), and FIG. 4B shows the picture of the hydrogel formed by adding 6.5 mg of 8 into 1.8 ml of water, corresponding to ˜0.36 wt % (2.2 mM) of the gelator and ˜23000 of water molecules/gelator molecule. 8 was unexpectedly potent (0.125 to 2 μg/ml, being 8 to 11 fold dilutions lower than the corresponding vancomycin) against VRE (2 vanA-positive Enterococcus faecalis, 4 vanA-positive E. faecium, 4 vanB-positive E. faecium). The strong tendency to self-assemble and the unexpected potency of 8 also lead us to speculate that 8 might aggregate into suprarnolecular structures at the cell surface when its local concentration is high.

### EXAMPLE 4

### Enzymatic Formation of Supramolecular Hydrogels

Recently, Hu and Messersmith (2003) reported using an enzyme to crosslink polymers to induce hydrogelation, and Lee et al. (2003) demonstrated using cells as the crosslinkers for polymers to promote gelation. Both methods are believed to be advantageous in the biomedical application of hydrogels. Similar methodologies, however, have yet to be explored with hydrogels formed by small molecules. The term “small molecules” means molecules without covalently linked repeating units and includes small peptides (e.g., derivatives of single amino acids, dipeptides, tripeptides, β-amino acids, and pentapetides, whereby the molecular weight of said derivatives are less than 3.0 kD). As used in the present disclosure, “small molecules” may be used interchangeably with “non-polymeric” molecules.

In the present invention, an enzymatic reaction was used to convert an ionic group on a derivative of an amino acid into a neutral group, which creates a small molecular hydrogelator and leads to the formation of a supramolecular hydrogel. This gelation process utilizes an alkaline phosphatase, one of the components of kinase/phosphatase switches that regulate protein activity, to dephosphorylate the PO₄³⁻ of N-(fluorenyl-methoxycarbonyl)tyrosine phosphate (9) under basic conditions. Unlike previously reported enzymatic gelation processes, this process, which involves bond breaking rather than bond formation, adjusts the balance of the hydrophobicity and hydrophilicity of the precursor, a simple amphiphilic derivative of amino acids, to yield a hydrogelator. Since dephosphorylation is a common, yet important, biological reaction existing in many organisms, its coupling with hydrogelation provides an advantageous way of generating and utilizing biomaterials based on supramolecular hydrogels.

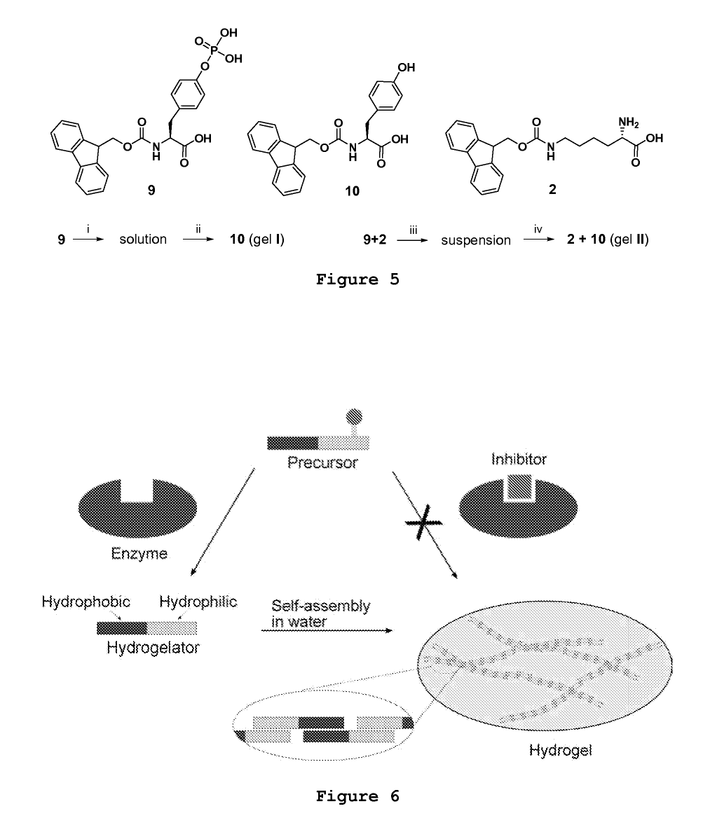

FIG. 5 illustrates two typical procedures for inducing gelation by dephosphorylation of 9. In the first case, 9 and one equivalent Na₂CO₃ is dissolved in a phosphate buffer (pH=9.6) to form a clear solution. The addition of alkaline phosphatase converts the solution of 9 into an opaque hydrogel of 10 with pH of 9.6 at 37° C. in 30 min.

In the second case, equal moles of 9 and 2 and two equivalents of Na₂CO₃ are mixed in the phosphate buffer (pH=9.6) to form a suspension upon gentle heating. The suspension is then added to the alkaline phosphatase and kept at ˜60° C. for three minutes. The suspension turns into a clear solution, which forms a clear hydrogel upon cooling to room temperature. When the same two procedures were repeated without the addition of the alkaline phosphatase, neither procedure led to the formation of hydrogels.

### EXAMPLE 5

### Using Supramolecular Hydrogels to Screen Enzyme Inhibitors

FIG. 6 illustrates the design of the visual assay. The precursor, which acts as the substrate of an enzyme, transforms into a hydrogelator when the enzyme catalyzes its conversion. Then, the self-assembly of the hydrogelators in water induces the formation of hydrogel. When inhibitors competitively bind with the active site of the enzyme and block the conversion of the precursor catalyzed by the enzyme, no hydrogel forms. Therefore, the macroscopic solution-to-gel transition (which can be observed visually) of the solution of the precursor reports the inactivation of the enzyme by the inhibitors.

This approach has a unique feature—it enlists water molecules as part of the reporting system. In addition, no spectrometer is required for observing the solution-to-gel phase transition. This simple and inexpensive method may be useful, not only for screening the inhibitors but also, for detecting the presence of enzymes when appropriate precursors are used. To verify the feasibility of the design shown in FIG. 6, a simple amino acid derivative (9), which can be converted into a hydrogelator (10) by dephosphorylation, was used to screen the inhibitors for an acid phosphatase.

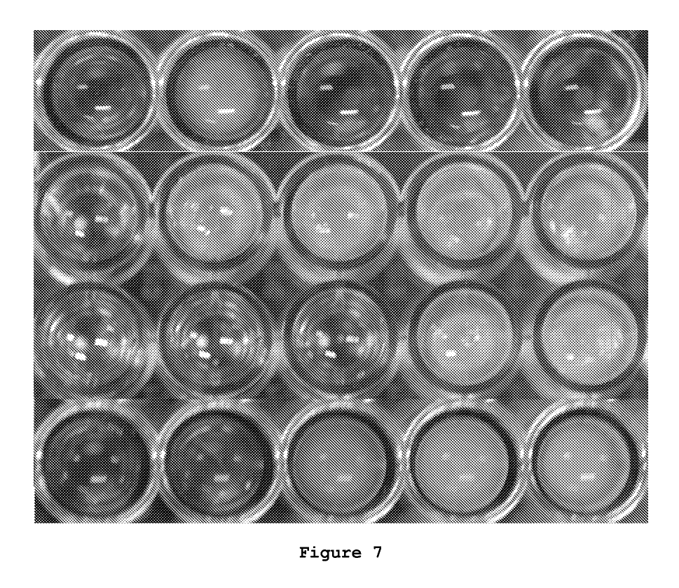

Since the acid phosphatase catalyzes the conversion of 9 to 10 and leads to hydrogelation at a pH=6.0 and 37° C., the event of hydrogelation can indicate the activity of inhibitors for the acid phosphatase itself. Pamidronate disodium, Zn²⁺, and sodium orthovanadate (Na₃VO₄) were chosen to estimate their minimum inhibition concentrations for the acid phosphatase. The three compounds were first mixed with the enzyme at a series of concentrations, respectively, followed by the addition of 9 to the solutions 10 minutes after mixing. After an additional 30 minutes of incubation, the solution-to-gel phase transition indicates the minimum inhibition concentration of the compounds. From the changes of rows 2, 3, and 4 in FIG. 7, the minimum inhibition concentrations of Pamidronate disodium, Zn²⁺, and sodium orthovanadate (Na₃VO₄) for the acid phosphatase were determined to be 33 mM, 0.33 mM, and 3.3 mM, respectively. This result corresponds closely to the literature values for this enzyme, thus validating our design.

### EXAMPLE 6

### Magnetoresponse of Supramolecular Hydrogels

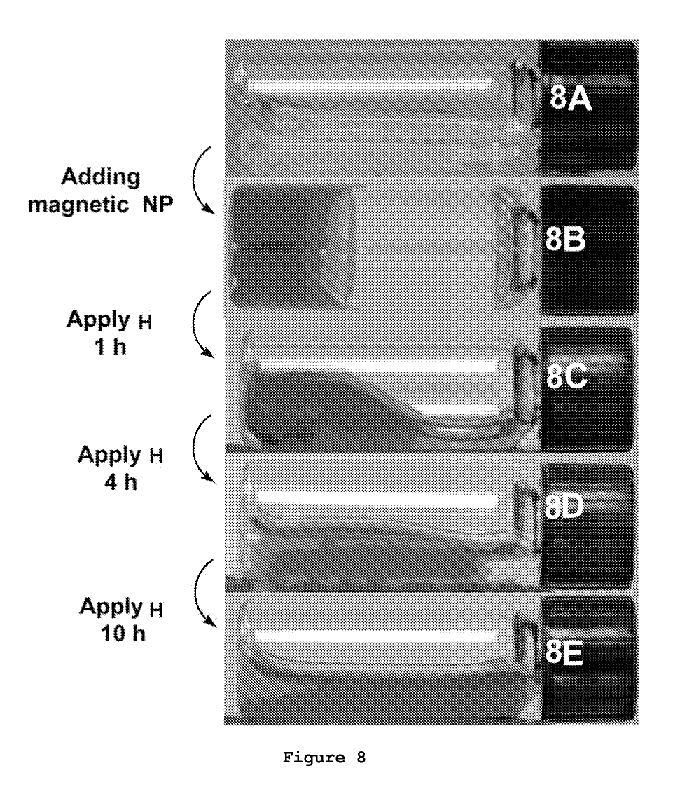

FIG. 8 shows the formation of the magnetic responsive hydrogel (FIG. 8B) after adding surface-modified magnetic nanoparticles into the solution of the diluted hydrogelator (FIG. 8A). After applying a small magnetic field to the hydrogel constantly for 10 hours (FIG. 8E), the hydrogel transforms into a solution and the aggregate of magnetic nanoparticles (for example, iron oxide). This process can be used to trigger the release of a drug from the hydrogel by a magnetic force.

### EXAMPLE 7

### Hydrogelators of Naphthalene-Containing Dipeptides

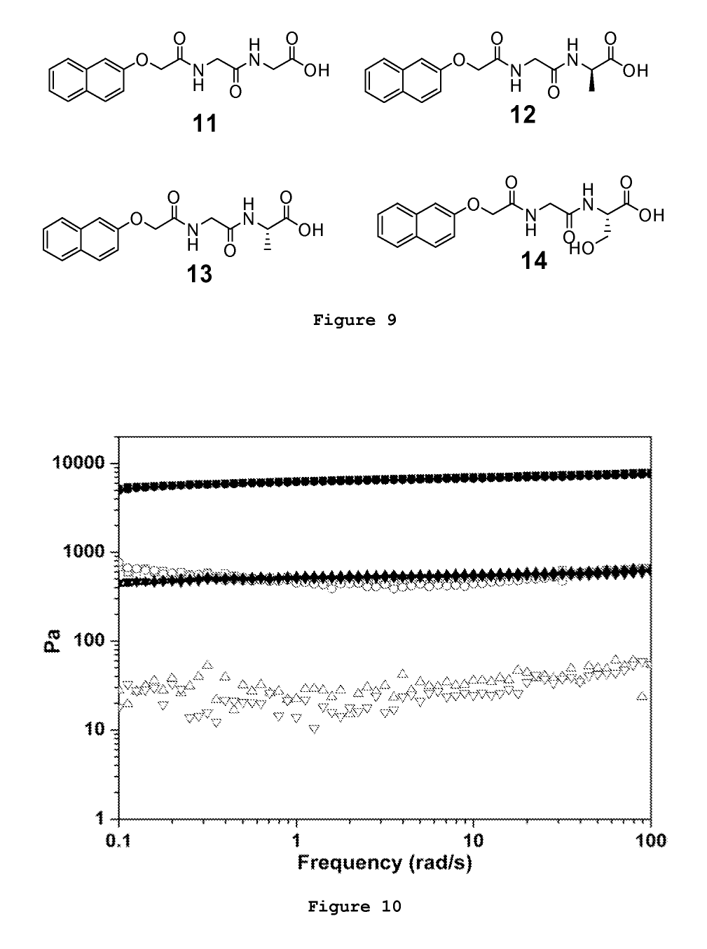

Hydrogelators can be made more biocompatible by containing a naphthalene group, a common fragment in drug molecules. FIG. 9 shows the chemical structures of the naphthalene-containing dipeptides that are hydrogelators. The syntheses of compounds 11, 12, 13, and 14 were based on 2-(naphthalen-2-yloxy)acetic acid. The syntheses of 11-14 were quite simple, just requiring the use of an active ester of N-hydroxy succinimine to react with different amino acids, and the overall yields were relatively high (60-80%).

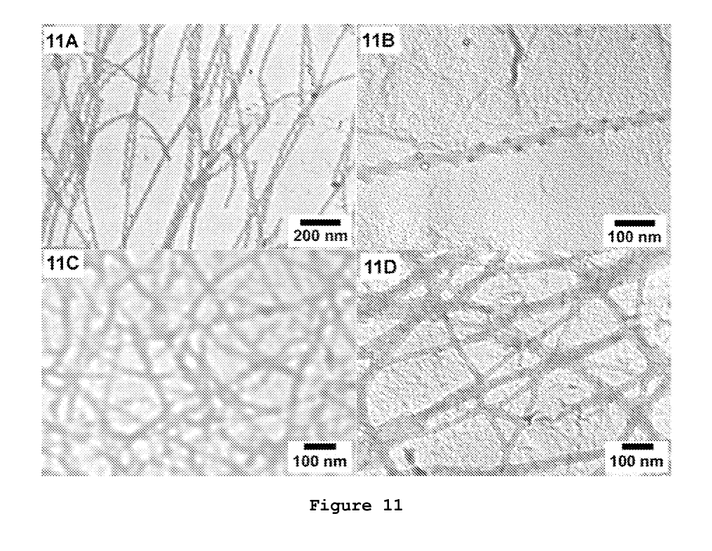

Compound 11-14 showed excellent abilities to gel water at pH˜2 and could form gels with concentrations of <0.10 wt %. Compounds 12 and 13 were the best gelators and could gel water at a concentration of 0.07 wt %. Compounds 11 and 14 exhibited similar behaviors of gelation to 2 and 3, except at higher concentrations ([11]=0.10 wt % and [14]=0.08 wt %). FIG. 10 shows the linear viscoelastic frequency sweep response of the four as-prepared hydrogels. All of them exhibited very weak frequency dependence from 0.1 to 100 rad/s, with G′ dominating G″, which means that they are effectively hydrogels. FIG. 11 displays the transmission electron micrographs (TEM) of the hydrogels, which reveals that the hydrogels made from 12 (FIG. 11B) or 13 (FIG. 11C) containing helical structures with very uniform size of about 30 nm and pitchs of about 60 nm. These results demonstrated that naphthalene moiety is an effective hydrogelation promoter. From these results, molecules bearing aromatic moieties (i.e., two or more benzene rings fused together) and di-, tri-, and tetrapeptides would be effective hydrogelators.

### EXAMPLE 8

### Hydrogelators of Pentapeptide Derivatives

In order to explore pentapeptide-based hydrogels as potential biomaterials, three aromatic moieties (pyrene (P), fluorene (F), and naphthalene (N)) were covalently linked to a series of pentapeptides: GAGAS, SEQ ID No. 1, (15), GVGPVP, SEQ ID No. 2, (16), VPGVG, SEQ ID No. 3, (17), VTEEI, SEQ ID No. 4 (18), VYGGG, SEQ ID No. 5, (19), and YGFGG, SEQ ID No. 6 (20). The balance of intermolecular aromatic-aromatic interactions and hydrogen bonds of these molecules can lead to their self-assemblies in water, which provide matrices of nanofibers for hydrogelation.

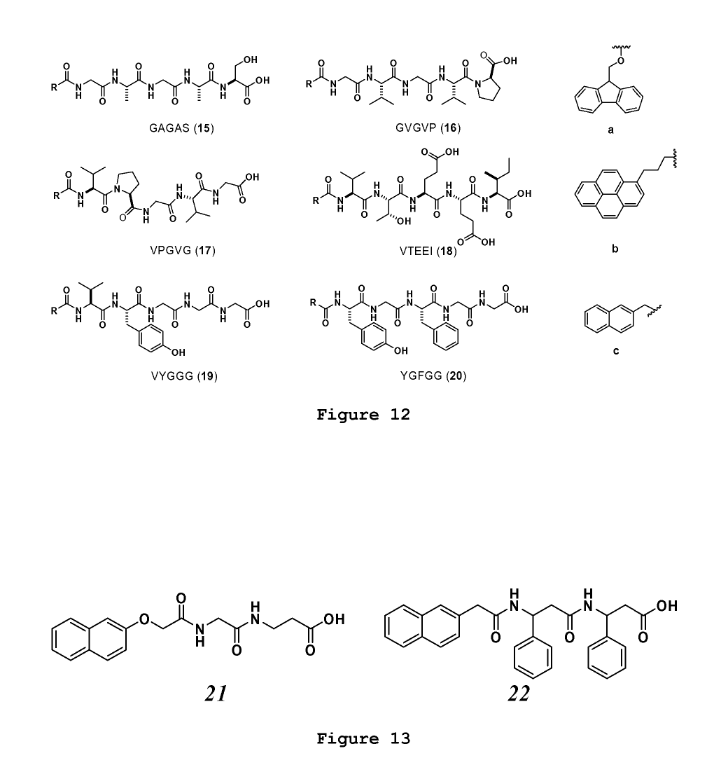

All the pentapeptides (structures shown in FIG. 12) were prepared by solid-phase synthesis using 2-chlorotrityl resin and the corresponding N^{α}-Fmoc protected amino acids with side chains properly protected by a t-butyl group. The first amino acid at C-terminal was loaded on the resin, followed by removal of the Fmoc group. Then the next Fmoc-protected amino acid was coupled with the free amino group using TBTU/HOBt as the coupling reagent. Finally, the N-terminus of the pentapeptides were either protected by Fmoc or coupled with 1-pyrenebutyric acid or 1-naphthalen acetic acid to afford the hydrophobic group. Upon completion of all the coupling, the pentapeptides were cleaved from the resin by trifluoroacetic acid (TFA) with 2.5% triisopropylsilane and 2.5% water as scavenger and purified by reverse phase HPLC. Gelation properties of the pentapeptides are shown in FIG. 15.

Most of the compounds can gel water under appropriate pH. When the pH becomes higher than the listed value, the gel tends to become a clear solution, while a lower pH always leads to precipitation rather than homogeneous gel formation. GAGAS (SEQ ID No. 1), the epitope with the least bulk side chains, appears to be quite hydrophilic in water. Naphthalene seems not to be hydrophobic enough to keep the hydrophobic/hydrophilic balance needed for Naph-GAGAS to gel water since Naph-GAGAS is soluble in water even under low pH and high concentration. With a more hydrophobic group, Fmoc-GAGAS and Pyrene-GAGAS become hydrogelators which can gel water under quite acidic conditions.

GVGVP (SEQ ID No. 2), with larger side-chains in valine and a proline at the end of the peptide chain, shows poor solubility in water. However, it is still not a good candidate as a hydrophilic tail in a hydrogelator. Only Pyrene-GVGVP can form gel easily. Hydrogel by Fmoc-GVGVP can be obtained by carefully adjusting the pH to 4.8, with the hydrogel not being thermal-reversible. Naph-GVGVP either dissolves in water at a pH higher than 4 or becomes a suspension at a lower pH. Upon heating, it also melts. All three compounds, with VPGVG as the hydrophilic part, fail to gel water at the tested condition. They all show sharp solubility changes with pH and low melting points.

VTEEI (SEQ ID No. 4), in which all the five amino acids have large side chains, shows a satisfactory ability to gel water when attached to Fmoc, pyrene or naphthalene. Notably, epitope VYGGG (SEQ ID No. 5), Fmoc, and naphthalene are appropriate hydrophobic groups for forming hydrogels while pyrene appears to be so hydrophobic that Pyrene-VYGGG is insoluble in water even under basic conditions. These examples demonstrate that pentapetides can be converted into excellent hydrogelators for generating supramolecular hydrogels as potential biomaterials. From these results, molecules having aromatic systems (aromatic moieties, i.e., two or more benzene rings that fused together) and pentapeptides or oligopeptides are effective hydrogelators.

### EXAMPLE 9

### Hydrogelators of β-Amino Acid Derivatives

Being used in vivo, oligopeptide-based scaffolds are biodegradable because proteolytic enzymes in biological systems will catalyze their hydrolysis (Seebach and Matthews, 1997). Such an inherent susceptibility towards enzymes shortens the in vivo lifetime of these peptide-based hydrogels, reduces their efficacy, and limits their scope of applications when long-term bioavailability is required. The disadvantage of proteolysis is a common feature for peptide-based therapeutic agents. Therefore, many efforts have focused on designing and synthesizing non-peptide molecules that mimic the functions of peptides or proteins to achieve prolonged or controlled stability and bioavailability of those molecules (Seebach and Matthews, 1997).

Among the peptidomimics (Giannis, 1993), β-peptides, which contain β-amino acids, have received intensive attention due to their improved biostability (Seebach and Matthews, 1997; Appella et al, 1996; Seebach et al., 1998; Hook et al., 2005; Martinek and Fulop, 2003; Porter et al., 2000). Despite the rapid progress in the designing and synthesis of β-peptides, the application of β-amino acids for controlling the bioavailability of supermolecular hydrogels remains unexplored since it is unknown if a β-amino acid derivative will act as a hydrogelator.

FIG. 13 illustrates the chemical structures of the two hydrogelators 21 and 22, which are dipeptidic mimics linked with naphthalene groups via amide bonds. The synthesis of both compounds is simple and straighforward: the N-hydroxy succinimide (NHS) activated ester of 2-(naphthalen-2-yloxy)acetic acid or 2-(naphthalen-2-yl)acetic acid react with glycine or β³-phenylalanine to afford 2-(2-(naphthalen-2-yloxy)acetamido)acetic acid or 3-(2-(naphthalen-2-yl)acetamido)-3-phenylpropanoic acid, respectively. The subsequent NHS assisted coupling gives 21 in 67% yield, and 22 in 72% yield.

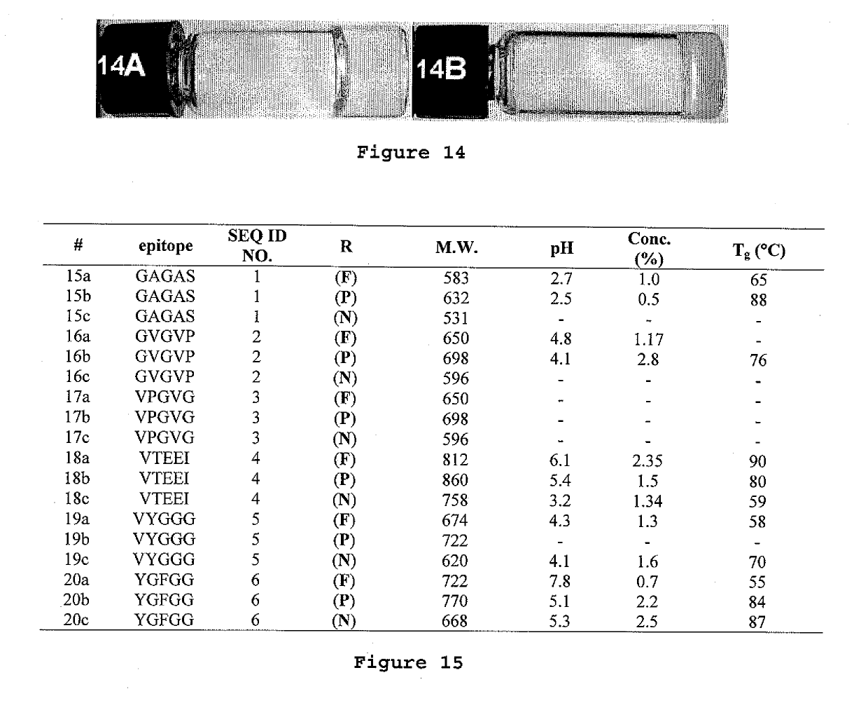

After 5 mg of 1 is suspended in 1.0 mL of water, the adjustment of the pH value of the suspension to 4.8 results in a clear solution, which provides a transparent hydrogel (FIG. 14A). Similarly, 5 mg of 2 in 1.0 mL of water also can form an slightly opaque hydrogel (FIG. 14B) by adjusting the pH or temperature. The confirmation of β-amino acids-based hydrogelators should provide a new way to tailor the stability of hydrogels in a biological enviroment and ultimately expand the ranges of applications of the hydrogens as biomaterials. From these results, molecules comprising β-amino acid derivatives and providing pi-pi interaction, hydrogen bonding, and other non-covalent interactions would be effective and stable hydrogelators.

### EXAMPLE 10

### Design and Synthesis of a β-Lactam Conjugate to Assay β-Lactam Resistant Bacteria

β-Lactam antibiotics (e.g., penicillins and cephalosporins), a major class of antimicrobial agents in clinical use for treating bacterial infections, rely on the strained β-lactam ring to react with penicillin-binding-proteins (PBPs) to inhibit cell-wall synthesis and growth of bacteria. β-Lactamases hydrolyze the four-member β-lactam ring and cause the most widespread antimicrobial drug resistance. Thus, it is essential to detect the presence of β-lactamases and screen their inhibitors. Although fluorescent (e.g., genotyping-based on polymerase chain reaction (PCR)) or calorimetric assays (e.g. using nitrocefin as indicator) are able to perform such tasks, a simple, rapid, and accurate assay is desirable because calorimetric assay fails in a colored medium and PCR remains costly and time consuming.

In the present invention, the inventors choose to use the event of hydrogelation to report the presence of β-lactamases because the formation of supramolecular hydrogels offers several advantages as an assay for an enzyme: (i) It is easy to determine a macroscopic change such as hydrogelation (even in a colored medium) by naked-eyes, thus eliminating the need of any instrument; (ii) an enzyme can catalyze either bond-formation or bond-cleavage to trigger hydrogelation, which makes this strategy suitable for a wide range of enzymes; and (iii) the hydrogel enlists water as part of the reporting system so that it can serve as a low-cost assay to be used in developing economies.

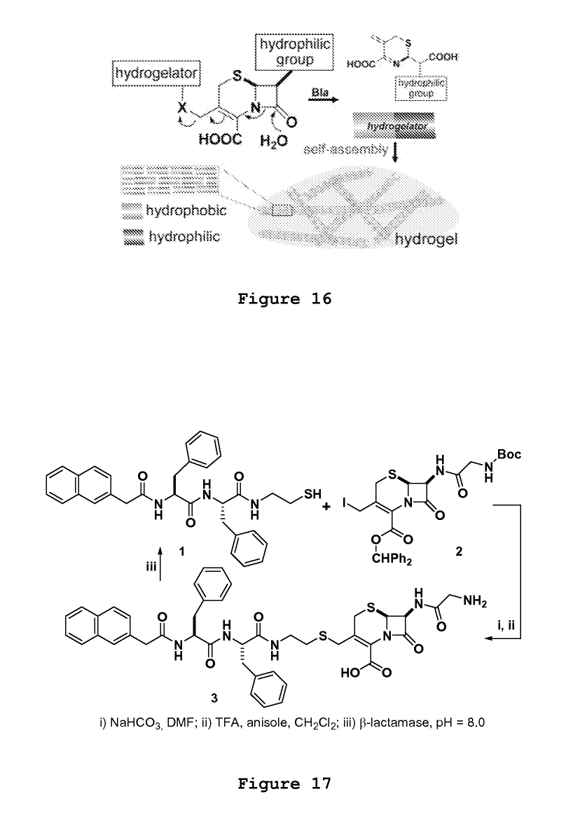

FIG. 16 outlines the general principle and molecular design for a β-lactamase catalyzed hydrogelation. Using the cephem nucleus as the linker, a hydrophilic group connects a hydrogelator to constitute the precursor, which is too soluble to form a hydrogel (i.e., the precursor supplies too little hydrophobic interaction to self-assemble into nanofibers that gel water). Upon the action of a β-lactamase, the β-lactam ring opens to release the hydrogelator, which self-assembles in water into nanofibers to afford a hydrogel. The key feature of the design is to use a β-lactamase to generate a hydrogelator.

FIG. 17 shows the actual structures and the synthesis of the molecules that employ the design in FIG. 16. An N-hydroxysuccinimide (NHS) activated napthalene-phe-phe (Nap-FF) reacts with 2-aminoethanethiol to yield an effective hydrogelator, which forms hydrogels at the concentration of 0.3 wt %. Following literature procedure, 7-amino-3-cholormethyl 3-cephem-4-carboxylic acid diphenyl-methyl ester hydrochloride (ACLH) was converted into 2. The nucleophilic substitution between 1 and 2 in a weak basic condition, followed by a simple deprotection (i.e., removal of Boc), creates the precursor 3 in a good yield (85.4%).

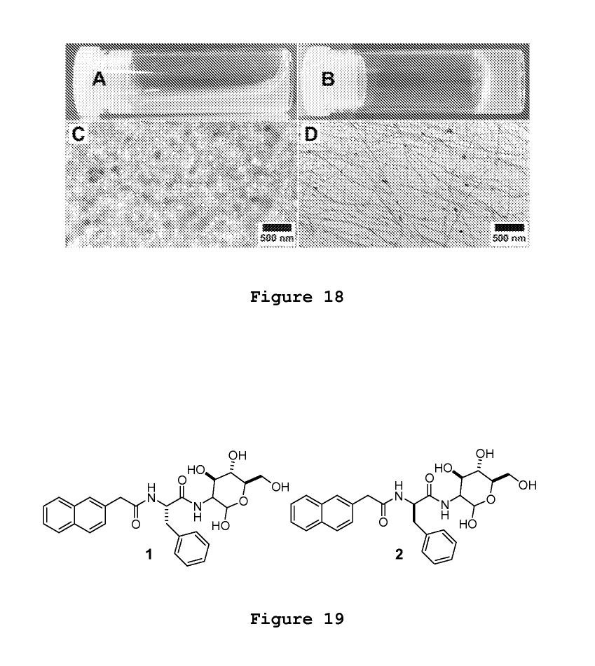

After obtaining the precursor 3, we tested if a β-lactamase would trigger hydrogelation. Precursor 3 (1.75 mg) dissolved in water (0.50 mL, pH=8.0) to form a viscous solution (FIG. 18A). Half hour after the addition of 0.55 mg of a β-lactamase (15-25 U/mg) to the solution at room temperature, the liquid turned into an slightly opaque hydrogel (gel I, FIG. 18B). HPLC test revealed that 49.0% of precursor 3 was transformed into 1 one hour after addition of β-lactmase. Rheological experiment (i.e., dynamic time sweep) confirmed that the solution of 3 is a Newtonian liquid and indicated that the hydrogelation started at about 22 minutes after addition of the β-lactamases. According to the TEM images shown in FIG. 18, the cyro-dried solution of precursor 3 is unable to exhibit a well-defined nanostructure (FIG. 18C), and the cyro-dried gel I showed nanofibrils with the diameters from 30 to 70 nm (FIG. 18D). It was also found that addition of precursor 3 into a solution of β-lactamase and its inhibitor (i.e., clavulanic acid) resulted in only 3.4% conversion of 3 to 1 after 12 hours (based on the HPLC test) and failed to yield a hydrogel. These results may lead to a convenient method to screen the inhibitor of β-lactamase by using enzymatic hydrogelation.

To evaluate whether precursor 3 would respond to β-lactamases in bacteria, solution of precursor 3 was treated with sonicated lysates of E. coli. As shown in Table 1, samples B, C, E, and F were the lysates containing different kinds of β-lactamases (CTX-M13, CTX-M14, SHV-1, and TEM-1, respectively), others are controls. Hydrogelation was triggered by the four kinds of β-lactamase. The HPLC traces clearly indicated effective conversion of precursor 3 to 1 (99.7%, 99.5%, 65.2%, 84.3% in samples B, C, E, F, respectively, but 5.8% in sample D and <0.5% on samples A, G) by adding different cell lysates. TEM images also showed self-assembled nanofibers in those four hydrogels resulted from the hydrolysis of 3 catalyzed by the β-lactamases. No hydrogelation was observed for sample D indicating that this gelation-based assay has a higher reporting threshold than the nitrocefin assay. This enzymatic hydrogelation-based assay thus provides a particularly useful reporting method for systems that have significant background activity that would cause false positive on nitrocefin assay. More completed conversion in samples B and C than in samples E and F also coincides with that CTX-M13 and CTX-M14 are β-lactam resistant bacteria. This observation may lead to an alternative approach to assay β-lactam resistant bacteria in a more specific way via tailoring the structure of the precursors.

In summary, it is demonstrated that β-lactamase is able to catalyze the formation of a supramolecular hydrogel. This approach, which involves the use of β-lactamase to control the self-assembly of small molecules, offers an alternative platform to study the inactivation of β-lactam antibiotics, provides an unique opportunity to generate nanostructures in regulated biological environment, and may lead to useful practical applications (e.g., selectively detecting β-lactam resistant bacteria in a clinical setting).

TABLE 1
Results of Adding Different Types of Cell Lysates
To Solutions of Precursor 3 (0.35 wt %)^{a)}

| Sample | Enzyme^{b)} | Gelation^{g)} | Conversion (%)^{h)} | Nitrocefin |
|---|---|---|---|---|
| A | C600^{d)} | − | <0.5 | − |
| B | CTX-M13^{c)} | + | 99.7 | + |
| C | CTX-M14^{c)} | + | 99.5 | + |
| D | JP995^{d)} | − | 5.8 | + |
| E | SHV-1^{e)} | + | 65.2 | + |
| F | TEM-1^{e)} | + | 84.3 | + |
| G | None^{f)} | − | <0.5 | − |

^{a)}Conducted as a blind test.
^{b)}Enzyme in 1.0 mL of the lysates of E. coli (10¹⁰ cells), except G.
^{c)}extended-spectrum β-lactamase (ESBL).
^{d)}A and D were β-lactamase negative E. coli controls.
^{e)}broad-spectrum β-lactamase.
^{f)}G contains only water.
^{g)}Gels form in less than 2 hours.
^{h)}Percentage of precursor 3 to form 1 after 6 hours.

### EXAMPLE 11

### Improving Wound-Healin by Carbohydrate-Based Hydrogelators

Glucosamine, a naturally occurring compound found in healthy cartilage, serves as a normal constituent of glycoaminoglycans in cartilage matrix and synovial fluid in the form of glucosamine sulfate, which strengthens cartilage and aids the synthesis of glycosaminoglycan. Therefore, glucosamine acts as one of the components in a widely used pain management for osteoarthritis patients and achieved moderate effectiveness. Glucosamine also plays a role in the process of wound healing, which has led to the successful demonstration that the dendrimer of glucosamine prevents the formation of scar tissues in a clinically relevant rabbit model. Apparently, the dendrimer of glucosamine inhibits Toll-like receptor 4 (TLR4) to achieve defined immuno-modulatory and antiangiogenic effects for synergistically preventing the formation scar tissue. The biological importance of glucosamine, the success of design and application of polyvalent glucosamine, and the successful generation and applications of low molecular weight gelators based on carbohydrates encourage us to incorporate glucosamine into supramolecular hydrogelators as the starting point towards self-assembled polyvalency of D-glucosamine for wound healing and other biomedical applications.

Despite intense research interests in glucosamine and the increased efforts on supramolecular gelators or self-assembled nanofibers, the use of glucosamine as a building block to generate supramolecular hydrogels remains unexplored, except Estroff and Hamilton (2004) suggested that the conformational rigidity of sugars plays an important role for hydrogelation via directing the intermolecular hydrogen-bond networks. Moreover, polymeric hydrogels that incorporate glucosamine or aminosugars exhibit increased adhesion with neural tissue of the host, improved vascularization, and enhanced infiltration of non-neuronal cells of the host. This observation suggests that glucosamine may exert similar beneficial effects to the supramolecular hydrogels and render them as a new type of biomaterials for applications in biomedicine.

It was found that the attachment of proper hydrophobic groups to the glucosamine is affords supramolecular hydrogelators with good biocompatibility. More importantly, the resulted hydrogel assisted wound healing and prevented formation of scar on a mouse model. The results of this work also supports the notion that self-assembly of bioactive molecules to form networks of nanofibers in hydrogel may offer a useful and effective way to generate biomaterials.

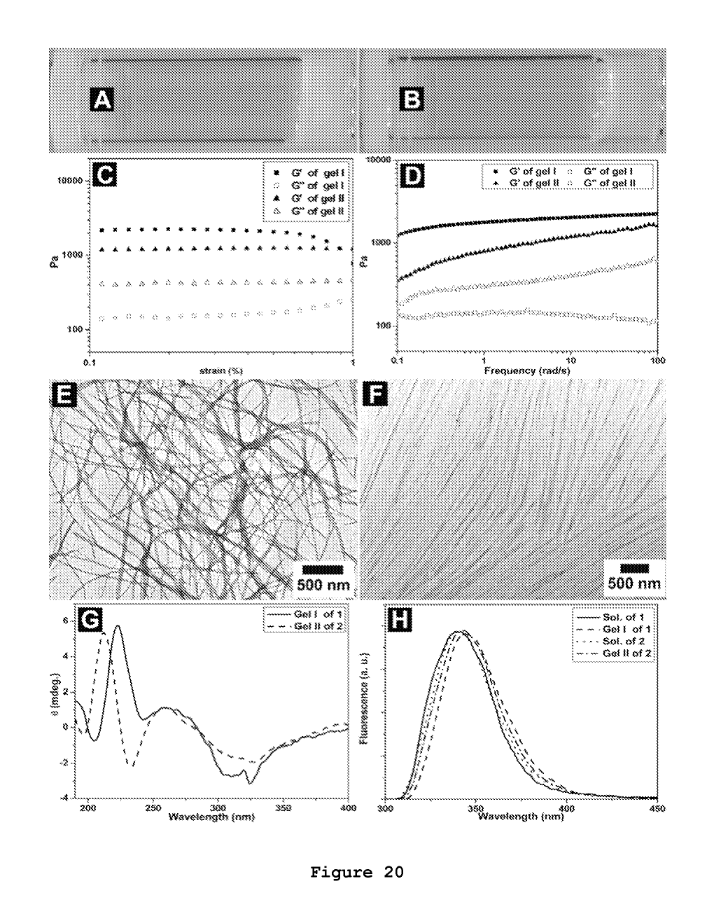

FIG. 19 illustrates the structures of two hydrogelators, which consist of D-glucosamine, L- or D-phenylalanine, and a naphthalene group. L-phenylalanine or D-phenylalanine reacted with N-hydroxy succinimide (NHS) activated ester of 2-(naphthalen-2-yloxy)acetic acid to afford (S)-2-(2-(naphthalen-2-yl)acetamido)-3-phenylpropanoic acid (3) and (R)-2-(2-(naphthalen-2-yl)acetamido)-3-phenylpropanoic acid (4), respectively. Then, NHS assisted coupling between 3 with D-glucosamine gave pure compound of 1 in 66% yield after HPLC, and coupling between 4 and D-glucosamine afforded pure compound of 2 in 63% yield after HPLC. Both 1 and 2 were effective hydrogelators. Typically, after 2 mg of 1 was suspended in 1.0 mL of water, the increase of temperature to 80° C. gave a clear solution. Cooling the solution to room temperature led to a slightly opaque hydrogel (Gel I, FIG. 20A). Similar procedure afforded the hydrogel of 2 (Gel II, FIG. 20B). The pH values of Gels I and II were around 7, and the hydrogels were stable at room temperature for several months. We also synthesized Nap-D-Glucosamine and Nap-L-Phe-L-Phe-D-Glucosamine, which fail to form supramolecular hydrogels. This result indicates that the balance between hydrophobicity and hydrophilicity is very important for a molecular hydrogelator.

FIGS. 20C and 20D show the rheological data of Gels I and II. Using the mode of dynamic strain sweep at the frequency of 10 rad/s, we determined the optimal conditions for the measurements of dynamic frequency sweep. As shown in FIG. 20C, the values of G′ and G″ kept constant from 0.1 to about 0.7% strain for Gel I and from 0.1 to 1.0% strain for Gel II. Both samples' values of G′ were larger than values of G″, indicating that both samples were viscoelastic. Although the value of G′ of Gel I was larger than that of Gel II, the range of plateau of Gel II was wider than that of Gel I, suggesting that Gel I was slightly more viscoelastic, but Gel II was more tolerant to external force.

Based on the above results, we measured the dynamic frequency sweep of both hydrogels at the strain of 0.4%. The values of their storage moduli (G′) exceeded that of their loss moduli (G″) by a factor of 10 (for Gel I) and 1.5 (for Gel II), indicating that these two samples were viscoelastic and behaved like a typical hydrogel. For Gel I, the value of G′ exhibited weak dependence on frequency (from 0.1 to 100 rad/s) at the stress above 1000 Pa; for Gel II, its value of G′ changed from about 200 Pa at low frequency (0.1 rad/s) to more than 1000 Pa at high frequency (100 rad/s). This observation indicates that the matrices of Gel I have a good tolerance to the change of external force.

To study the microstructure of Gels I and II, we obtained transmission electron micrograph (TEM) images of the hydrogels. As shown in FIG. 20E, irregular small ribbons formed large bundles and tangled with each other in Gel I. We also observed a small amount of helical fibers with width range from 27 to 55 nm in Gel I. For Gel II (FIG. 20F), small rigid ribbons with width of 35-50 nm form well-distributed matrices. The sizes of the ribbons in Gel II were more uniform than those in Gel I. The density of nanostructures in Gel I was higher than that in Gel II, which accounts for a slightly larger value of G′ of Gel I than that of Gel II. The different morphologies in both gels are likely resulted from their different structures because the concentration of 1 or 2 was the same in their corresponding hydrogels and the only difference was the configuration of phenylalanine (L- for 1 and D- for 2). According to TEM images, compound 1 with an L-phenylalanine had a more tendency to aggregate to form larger bundles and a more crosslinked network.

The circular dichroism (CD) and fluorescence spectra of the hydrogels also help further understanding the molecular arrangements in Gels I and II. As shown in FIG. 20G, the peak at 191 nm and the trough at 205 nm in Gel I were resulted from exciton splitting of the peptide π-π* transition, while the peak at 222 nm was due to the peptide n-π* transition. In Gel II, the peptide π-π* and n-π* transition bands appear at 195 nm and 212 nm, respectively. The peaks at around 218 nm indicated unordered conformations of peptide bonds of both compounds (1 and 2) in their gel phases. These CD signals (below 240 nm) shared common features with the CD of β-sheet of a polypeptide, suggesting that the self-assembly of the hydrogelators leads to a β-sheet like superstructure. We also observed a broad positive peak centered at about 272 nm (nπ* of aromatic parts) and a broad negative peak centered at about 315 nm (ππ* of aromatic parts). The CD signals of Gels I and II exhibited similar shapes, suggesting that they were mainly induced by the D-glucosamine.

FIG. 20H shows the emission spectra of both 1 and 2 in solution and gel phases. Both compounds exhibited broad peaks centered at 340 nm in their corresponding solution phase. In their gel phases, the peaks showed slightly red shifts (to 347 nm for Gel I and to 343 nm for Gel II). These small red shifts indicated the lack of efficient π-π stacking of naphthalene groups of both compounds in their gel phases. The observations of a slightly bigger red shift and a higher shoulder peak at 375 nm in Gel I than those of Gel II correlated well with the results obtained from Theological measurements (higher elasticity or bigger G′ value for Gel I) and TEM images (more entangled fibers in Gel I).

After characterizing the physiochemical properties of the hydrogels, we evaluated their biocompatibility, one of the major requirements for the application of the hydrogels. The cytotoxicity assay of 3-(4,5-dimethylthiazol-2-yl)-2,5-diphenyl-tetra-zolium bromide (MTT) indicated that 73.8% and 79.0% of HeLa cells survived in 100 μM of 1 and 2 at 24 h, respectively. Based on this result, we chose Gel II to test its ability to reduce formation of scar tissue at the wound site on the mice wound model (Hirobe, 1988) since 2 is more biocompatible than 1. Subsequently, we tested whether the hydrogel would improve the wound healing using the following protocol: Six (6) Balb/C mice, aged 6 weeks were randomly divided into two groups viz: treatment and control. A cut (7 mm long, 2 mm wide, and 2 mm deep) was made on the middorsal skin of each mouse. After 30 seconds, 1 mL (2 mg of 2 in PBS) of Gel II was applied on the cut (a liquid bandage was also used to fix the hydrogel). For the negative control, only a liquid bandage was applied on the wounds. No clinical, hematological or biochemical (including blood glucose) toxicity were observed, and there was no local or systemic bacterial, viral, or fungal infections in both two groups treated over 18 days.

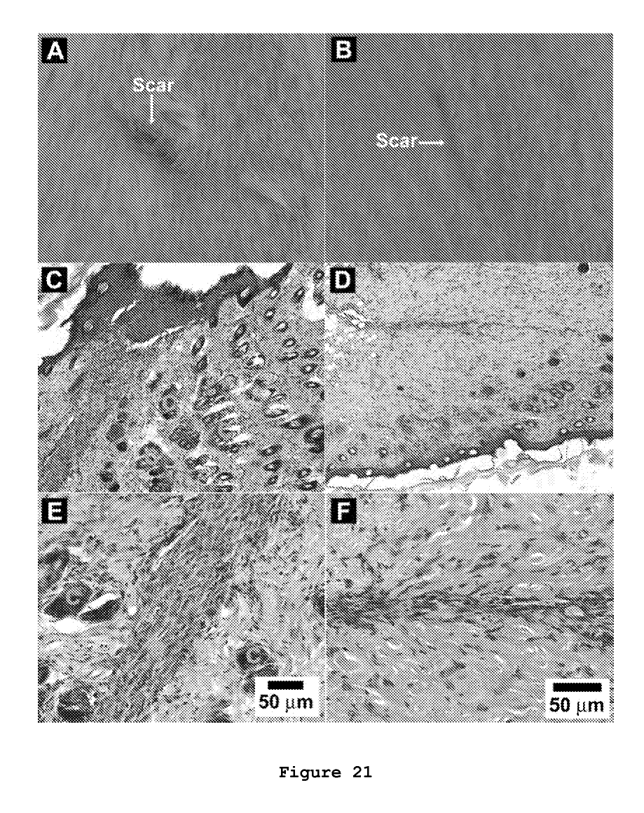

On inspection, the mice treated with Gel II exhibited a much faster wound healing process and smaller scars than those of control group at day 6 (FIG. 21A-B). Histological examination of the skins at the wound site also showed that higher density of fibroblasts (scar tissues) was presented in the skin of the mice in the control group. Large amount of keratinocytes migrated to the extracellular matrix (ECM) near the scar tissues (FIG. 21C), indicating that on day 6 the wound on the untreated mice was at the re-epithelialization phase, one of the five typical phases of wound healing. On the contrary, there was minimal formation of scar tissue at the wound site of the Gel II-treated mice on day 6. Large amount of ECM forms between the fibroblasts and the keratinocytes, which indicated the Gel II treated mice were at the later matrix deposition phase of wound healing (FIG. 21D). These results are consistent with the appearance of skin of the wound site after healing.

In summary, based on a biologically important aminosaccharide derivative, we successfully synthesized two novel small molecule hydrogelators, which form biocompatible and stable hydrogels. The mice with wounds on their back recovered more rapidly when treated with one of the hydrogels than those without the treatment. This result indicates that the biomaterial reported herein can be developed into a promising candidate for wound healing. Further work will focus on studying the detailed relationship between self-assembly of the glucosamine-based hydrogelators and the beneficial effects of the hydrogels for other biomedical applications. From these results, we derive structural features (i.e. aromatic system to provide pi-pi interaction, and amino acid and carbohydrate to provide other non-covalent interactions) that can be generally applied to other carbohydrate-based hydrogelators.

### EXAMPLE 12

### Unnatural Amino Acid-Based Hydrogelators for Controlled Drug Release

For long-term in vivo applications (e.g., controlled drug release), the supramolecular hydrogels should possess long-term stability and resist to various digestive enzymes in the biological system. The present invention develops supramolecular hdyrogelators based on unnatural amino acids (i.e., amino acids except the 20 natural ones) and demonstrates that β-peptide derivatives gel water efficiently.

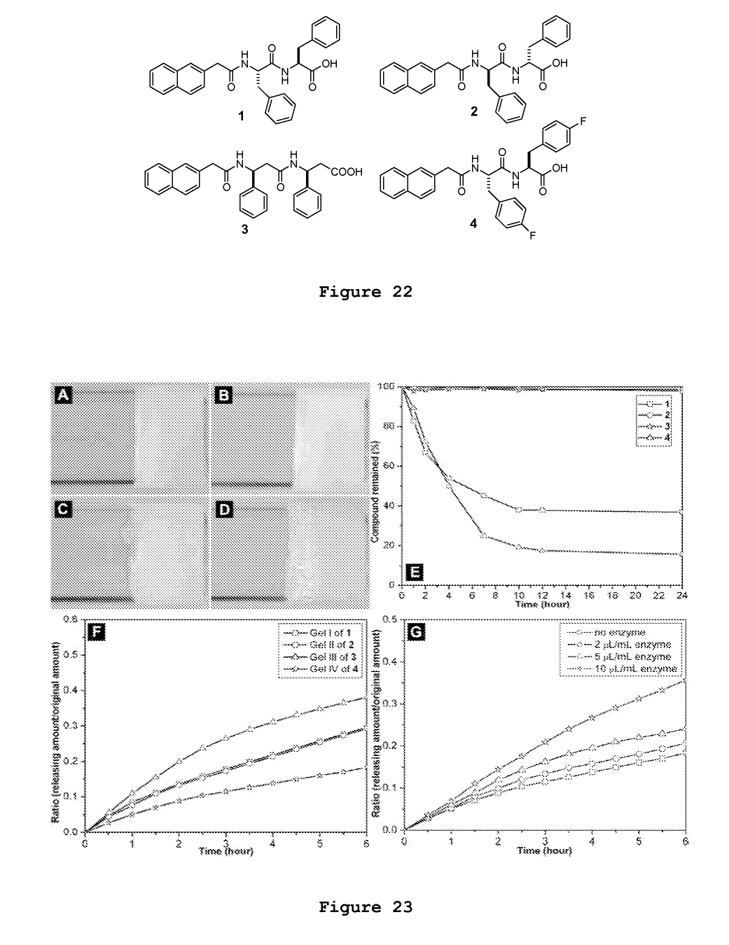

Despite the large pool of unnatural amino acids offers a range of possibilities for the exploration of supramolecular hydrogels, only few reports described the hydrogelators based on unnatural amino acid. In the present invention, three new unnatural amino acid-based hydrogelators were reported that share structural similarity with phe-phe dipeptide, a well-established small molecule that prones to self-assembly in water (see FIG. 22). Among them, 2 and 3 show excellent resistance to digestion catalyzed by proteinase K, an enzyme that catalyzes the hydrolysis of a broad spectrum of peptides. This finding suggests that unnatural amino acid-based molecular hydrogels may serve as biomaterials with good stability in vivo and may lead to potential applications in controlled drug release.

Based on the results that Nap-L-Phe-L-Phe and racemic Nap-β³-HPhg-β³-HPhg are efficient molecular hydrogelators, we choose the unnatural amino acids of D-phenylalanine (D-Phe), s-β³-Hphenylglycine (s-β³-HPhg), and L-4-fluorophenylalanine (L-fPhe) to make the dipeptide derivatives (see FIG. 22). The synthesis of the four compounds is easy and straightforward. The N-hydroxyl succinimide (NHS)-activated ester of 2-(naphthalen-2-yloxy)acetic acid reacted with one equivalent of corresponding amino acid to afford an amino acid derivative with naphthalene group and terminated with carboxylic acid, NHS activated the carboxylic acid to couple with one equiv. of the amino acid and to produce the compounds shown in FIG. 22. Each of the four compounds formed hydrogels. Typically, 10 mg of the compound dissolved readily in 1.0 mL of water at pH=10. The hydrogel formed upon carefully adjusting the pH of the solution to a certain value (˜7.5 for 1 and 2, ˜7.1 for 3, and ˜7.8 for 4). The hydrogels were stable at room temperature for at least one month.

Transmission electron micrograph (TEM) images of the cyro-dried gels (Gel I for 1, Gel II for 2, Gel III for 3, and Gel IV for 4) revealed that Gel I and Gel II showed similar morphologies, long nanofibrils with length over 10 μm and width about 50 nm. Gel III contained irregular fibers with widths ranging from 30 nm to about 250 nm, and the fibers showed strong tendency to aggregate into bundles and leave large pores in the matrix of the gel. Gel IV consisted of fibrils in much higher density than those in other three gels. The fibrils also had wide size distribution (20 to 150 nm) tangle with each other to form the three dimensional network.

After using MTT assay to verify the biocompatibility of the hydrogelators of 1-4, we evaluated the stability of the hydrogelators by incubating them with proteinase K at 37° C. in HEPES buffer solutions. As shown in FIG. 23, compound 2 and 3 showed strong resistance to enzymatic digestion, indicated by that their quantities remained almost the same as their original ones after incubation for 24 hours. On the contrary, 1 and 4 hydrolyzed easily in the presence of proteinase K: only 37% of 1 and 16% of 4 remained in solutions after being incubated for 24 hours with proteinase K, respectively. This result suggests that the gels formed by 2 or 3 could serve as potential candidates of biomaterials that require long-term stability.

Using folic acid, whose absorbance peak at about 360 nm (away from the absorption of the naphthalene group at 272 nm), as the model compound, we tested the hydrogels as the medium for controlled drug release. As shown in FIG. 23F, the release of folic acid was more rapidly in initial experimental time (0-2 hour) than that in later experiment time (2-6 hour). After 6 hours' incubation, 30%, 29%, 38%, and 18% of folic acid were released from the matrix of Gel I, Gel II, Gel III, and Gel IV, respectively. The released amount of folic acid agrees with the density of the nanofibers in the hydrogels (as indicated by the TEM images), that is, folic acid was released more rapidly from a gel with larger pores. FIG. 23 shows the optical images of four kinds of hydrogels six hours after the release of folic acid, the intensity of the yellow color (shows the amount of folic acid remained in the gel) in four gels follows the order of Gel IV>Gel I˜Gel II>Gel III. This result also matches well with the data in FIG. 23F. Because proteinase K catalyzes the hydrolysis of 4, the release of folic acid from Gel IV (formed by 4) could be controlled by adding different amount of the enzyme to the gel containing folic acid. As shown in FIG. 23G, the folic acid can be released more rapidly from the gel with higher concentration of proteinase K.

In summary, we synthesized a new type of low molecular weight hydrogelators based on unnatural amino acid, which afford hydrogels with long term stability for possible controlled drug release. Further work will focus on the in vivo drug controlled release from these novel molecular hydrogels. From these results, we derive general structural features (i.e. aromatic system to provide pi-pi interaction, and D-amino acids or fluorinated amino acids to provide other non-covalent interactions) of unnatural amino acid-based hydrogelators.

### EXAMPLE 13

### Hydrogelators of β-Amino Acid Derivatives

One approach to prolong the bioavailability of the hydrogelators is to introduce β-amino acid or β-peptide motif into the hydrogelators because many works have confirmed the resistances of β-peptides toward to a variety of peptidases, and β-peptides or similar peptidomimics could form more stable secondary structures than α-peptides. Except Gellman and co-workers have developed helical β-peptides that could self-assemble into lyotropic liquid crystals, and we showed that β-amino acid derivatives form supramolecular hydrogels, whether an enzyme catalyzes the formation of the β-amino acid-containing hydrogels and the biostability of the hydrogels have yet to be examined.

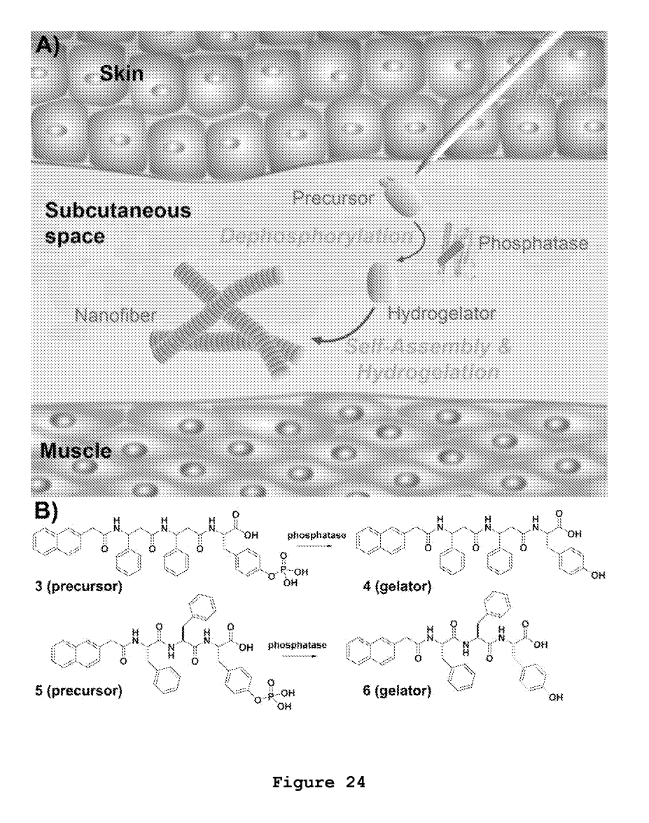

Based on the knowledge that Nap-β³-HPhg-β³-HPhg (1) is a hydrogelator, we design a chimera of tripeptide derivative (3) that consists of two β-amino acids (i.e., β³-homophenylglycine) and one α-amino acid residue (i.e., tyrosine phosphate) to evaluate whether it undergoes enzymatic hydrogelation in vitro and in vivo (see FIG. 24). While the tyrosine phosphate makes the precursor susceptible to the enzyme, the dipeptide segment of the β-amino acid would confer excellent stability of the resulted hydrogelator in biological environment. Upon being treated with acid phosphatase, 3 transforms into 4, which self-assembles into nanofibers and results in a supramolecular hydrogel in aqueous solution or complex fluids such as blood or cytoplasm. Moreover, in vivo experiment revealed that the hydrogels formed by β-amino acid derivatives had a longer lifetime than that of hydrogels formed by β-amino acid derivatives. Being the first demonstration of enzymatic formation and the first evaluation of bioavailability and biostability of supramolecular hydrogels constituted of β-amino acid derivatives, this result suggests that supramolecular hydrogels formed by β-amino acid derivatives could evolve into promising candidates for biomedical applications when long-term stability is required.

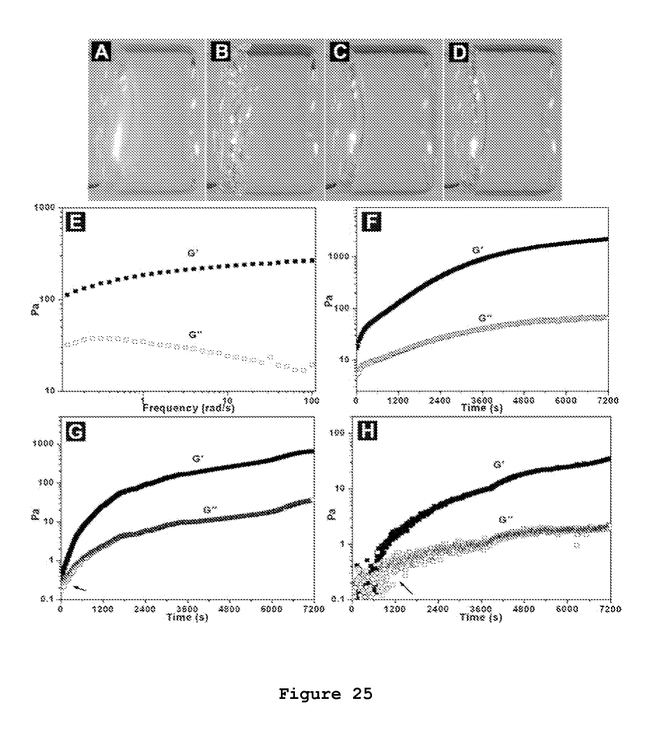

After being activated by N-hydroxysuccinimide (NHS), Nap-s-β³-HPhg-s-β³-Phg (2) reacted with O-phospho-tyrosine in the mixture of water and acetone (pH˜7.8) to afford the crude product of 3, which is further purified by liquid chromatography. 3 formed a slightly opaque hydrogel (Gel I in FIG. 25A) at the concentration of 0.5 wt % (6.91 mM) and pH of 1.5. 3, however, failed to gel water at the pH>3.0. Therefore, we choose to carry out enzymatic reactions at pH of 4.8 because the enzyme of acid phosphatase also works most efficiently at this pH. We choose different concentrations of the acid phosphatase (5.88, 2.94, and 1.47 U/mL) to catalyze the enzymatic reactions of 3 (2 mg) in 0.4 mL aqueous solution. Hydrogelation happened in all three samples (FIG. 25), but each took different time (Table 2). As expected, the least amount of the enzyme (1.47 U/mL) took the longest time (30 minutes) to achieve hydrogelation. Using more enzymes, the times needed for hydrogelation were shorter, which were about 10 and 2 minutes when the concentrations of enzyme were 2.94 and 5.88 U/mL, respectively. These results confirm that the amount of the enzyme easily controls the rate of hydrogelation, thus offering a convenient strategy to tailor the hydrogelation process for various applications.

TABLE 2
Parameters of Hydrogelation

| Gel # | [Enzyme](U/mL)^{a} | pH^{b} | t (min)^{c} | G′ (Pa)^{d} |
|---|---|---|---|---|
| I | 0 | 1.5 | | 200 |
| II | 5.88 | 4.8 | 2 | 4000 |
| III | 2.94 | 4.8 | 10 | 900 |
| IV | 1.47 | 4.8 | 30 | 300 |

^{a}The concentrations of acid phosphatase used to trigger the hydrogelation
^{b}pH values of the gels
^{c}time needed for gelation (observed by ”inversion” test)
^{d}the final values of storage moduli (G′) 72 hours after gelation.

The process of hydrogelation also directly affects the viscoelastic behavior of the resulted hydrogels. FIG. 25 shows the linear viscoelastic frequency sweep response of Gel I formed by the adjustment of pH and the dynamic time sweeps for studying the enzymatically-formed hydrogels (Gels II, III, and IV). Gel I exhibited a weak frequency dependence from 0.1 to 100 rad/s, with G′ dominating G″, suggesting that the sample was a hydrogel. Both values of G′ and G″ were lower than 200 Pa, indicating a weak network as the matrices of Gel I. FIG. 25F shows that the addition of enzyme (5.88 U/ml) to the aqueous solution of 3 (6.91 mM) triggered immediate hydrogelation (i.e., the value of G′ dominating that of G″ at the beginning of Theological experiment) to afford Gel II. FIG. 25G indicates that the hydrogelation still happened rather rapidly (within 1 minute) to afford Gel III when the concentration of enzyme was 2.94 U/mL. FIG. 25H shows the value of G′ dominating that of G″ in 20 minutes, confirming that further decreasing the amount of the enzyme (1.47 U/mL) resulted in slower hydrogelation to give Gel IV, The increases of G′ and G″ over time (FIG. 25F, G, H) indicate that the enzyme continuously converted the precursor 3 to the hydrogelator 4 and made more nanofibers. Although the values of G′ of the three enzymatically-formed hydrogels approached plateaus within two hours, HPLC analysis revealed that, two hours after adding the enzyme, 84.4%, 61.6%, and 40.8% of 3 transform into 4 in Gels II, III, IV, respectively. To further evaluate the influence of the amount of enzyme on the viscoelasticity, we measured the rheological properties of the three hydrogels 72 hours after the additions of enzyme (i.e., all the precursors converted into the hydrogelators according HPLC analysis). As shown in Table 2, the values of G′ of three gels were nearly 4000 Pa for Gel I, about 900 Pa for Gel II, and 300 Pa for Gel III. Though the three hydrogels contained the same amount of compound 4, their mechanical properties differ, and the hydrogel formed more rapidly exhibited a larger value of G′, implying that the ratio between the enzyme and the precursor could modulate the mechanical properties of the resulting hydrogels.

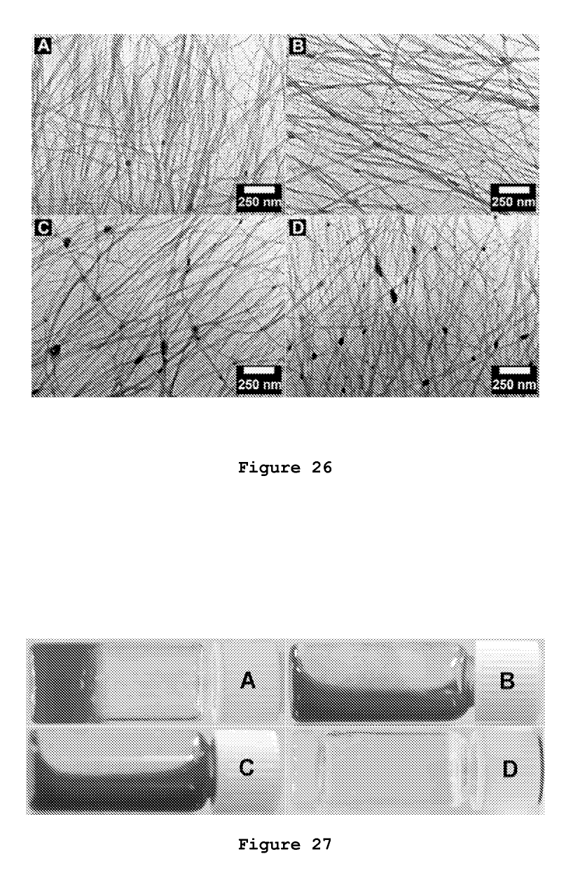

The difference in mechanical behavior correlates well with the microstructure of the hydrogels. As shown in their transmission electron micrograph (TEM) images (FIG. 26), the fibrils of Gel I (formed by changing pH) and that of other three gels (formed by enzymatic catalysis) were quite different: Gel I consisted of long and irregular fibers (ranging from 18 to 55 nm) and fibrous bundles (some were larger than 100 nm). Unlike Gel I, fibrils in the enzymatically-formed hydrogels were more uniform (20 to 40 nm, 25 to 37 nm, and 25 to 33 nm for Gels II, III, and IV, respectively) and exhibited high densities, likely due to different kinetics of hydrogelation. The change of pH allowed a large amount of hydrogelators to form almost instantaneously, affording fibrils with various widths and bundles of the fibrils. But each enzyme molecule created hydrogelators one by one, thus yielding more uniformed nanofibrils. The above hypothesis (or mechanism) matches well with the TEM results—the fibrils became more uniform when the enzymatic reaction was catalyzed by less amount of enzyme. Although the sizes of fibrils were more uniform in Gel III and Gel IV, their fibrils were thinner than the fibrils in Gel II, thus explaining the better mechanical properties of Gel II. The above results clearly indicate that the kinetics of hydrogelation (regulated by an enzyme) strongly influence the morphology of resulting nanofibers in the hydrogels.

After studying enzymatic hydrogelation of 3 in buffer solution, we also investigated whether enzymatic hydrogelation of 3 would proceed in more challenging conditions—i.e., in blood or cytoplasm. As shown in FIG. 27A, a hydrogel formed in about half an hour after the mixing of 0.3 mL of blood (from rabbit), 0.2 mL of the PBS solution containing 1.0 wt % of 3, and 10 μL of alkali phosphatase. No gelation of blood occurred without either the alkali phosphatase (FIG. 27B) or compound 3 (FIG. 27C). The time of gelation in blood was longer than the enzymatic hydrogelation in PBS buffer solutions, probably due to the high complexity (i.e., the components in blood compete with 3 for enzymatic dephosphorylation) and high viscosity of blood, which decreases the rate of enzymatic reaction. We also added the solution containing 3 (1.0 wt % of 3 in PBS) to broken Hela cells (1.0×10⁶). As shown in FIG. 27D, an opaque hydrogel formed immediately after adding 10 μL of alkali phosphatase into the mixture. Both results confirm that enzymatic hydrogelation of the β-peptide derivative proceeds in complex biological fluids containing various other enzymes and proteins, promising a simple strategy to detect enzymes (e.g., phosphatases) directly in complex or color fluids (e.g., blood) without any pretreatment.

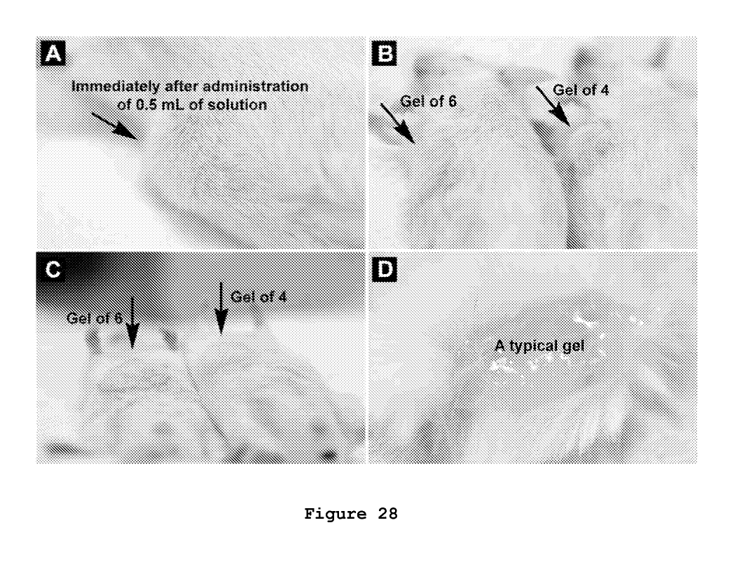

To further evaluate the biostability of 3, we synthesized compound 5 (see FIG. 24; a α-peptide analog of 3) and conducted the experiment to study/compare the gelation abilities and biostabilities of 3 and 5 in vivo. Both 3 and 5 formed opaque solutions in PBS buffer (pH=7.4) at the concentration of 0.8 wt %. To allow the gels to form within 5 minutes and minimize the diffusion of 3 or 5 in vivo, we chose 200 U/mL as the concentration of the phosphatase. After adding the same amount of alkali phosphatase to the solution of either 3 or 5, we immediately injected the solutions (0.5 mL) subcutaneously to a mouse. A small lump appeared at the site of injection (FIG. 28A), and it decreased over time probably due to diffusion and/or degradation of the compounds. Comparing the mice administrated with compounds 3 and 5 (FIG. 28B and C), the size of lump formed by 4 (β-peptide hydrogelator) decreased slower than that formed by compound 6 (α-peptide hydrogelator). Moreover, the lump at the injection site of β-peptide derivatives (3/4) was always larger than that of α-peptide derivatives (5/6) during the whole experiment. The above observations suggest that the enzymatically-formed hydrogels of β-peptide have longer life times in vivo than those formed by α-peptides.

HPLC analysis of the hydrogels at the injection sites (e.g., FIG. 28D) revealed enzymatic conversion of 3 and 5. The conversion of 5 to 6 was much faster than that of 3 to 4—56.7% of 5 changes to 6, but only 19.5% of 3 to 4 one hour after injection, which indicated that 5, as an α-peptide derivative, served as a better substrate for the phosphatase than 3 does. More than 90% of 3 or 5 transformed to the corresponding hydrogelators after 7 hours. Comparing the hydrogels formed by α- or β-peptide derivatives, we found that the diffusion or digestion of α-peptide derivatives was much faster than those of β-peptide derivatives. Only 72% of the α-peptide derivatives (5 and 6) remained at the injection site one hour after injection, and they disappeared almost completely after 24 hours (vs. the initial amount of 5). For the β-peptide derivatives, 94% of the compounds (3 and 4) remained at the injection site one hour after injection, 44% of compound 4 after 24 hours, and 21% of compound 4 after 72 hours (vs. the initial amount of 3). Based on the above results, the half-life (the time when half of the compounds disappeared from the injection site) of both samples were about 2.3 hours and 15 hours for 5 and 3, respectively, These results match well with the optical images in FIG. 28B and 28C and indicate that the molecular hydrogels formed by β-peptide derivatives could be useful biomaterials for long-term biostability.

We also examined in vivo cytotoxicity of the hydrogel by monitoring the weight change of the mice after injecting 3 and the enzyme into them. The mice that received subcutaneous injection of 3 (0.5 mL, 0.8 wt %) lost body weight in the first day (0.80 g, 4.0% decrease), and so did the mice in the control group (0.06 g, 0.3% decrease). The two groups of mice both started to gain body weight after the second day. A slightly more weight loss in day one of the mice administrated with the hydrogels than those injected with just saline suggests that subcutaneous administration of 3 and enzyme at the experimental dosage results in a little acute toxicity to the mice. The mice recovered to normal stage and started to gain body weights after the second day, suggesting that 4 was more biocompatible than 3.

In summary, we demonstrated that β-amino acid derivatives can serve as the substrate of an enzyme and afford hydrogels of longer biostability than that of α-amino acid derivative-hydrogels. Since the rapid development β-peptides that mimic the functions of α-peptides have already led to a few bioactive β-peptides recently, the exploration of enzyme-trigged β-peptide hydrogels offers a new opportunity to develop β-peptide-based (or other peptidomimics) biomaterials for biomedical applications.

### EXAMPLE 14

### Supramolecular Hydrogels-Encapsulated Active Center as an Artificial Enzyme

One of the Holy Grails in chemistry is an artificial enzyme that mimics the functions of enzymes by using systems simpler than proteins. The major efforts of the development of artificial enzymes have concentrated on designing simple molecular systems that reproduce characteristics of enzymatic reactions such as substrates binding, large rate acceleration under mild conditions, and high selectivity. The intensive development on artificial enzymes and the rapid progress in supramolecular hydrogels prompt us to evaluate whether supramolecular hydrogels will improve the activity of artificial enzymes for catalyzing reactions in water or in organic media. To demonstrate the concept, we choose self-assembled nanofibers of amino acids to act as a protein-like structure and hemin as the active sites to mimic peroxidase.

Heme peroxidases are ubiquitous enzymes that catalyze oxidation of a broad range of organic or inorganic substrates by hydrogen peroxide or by organic peroxides. Peroxidases, however, have shortcomings such as high costs, instability in solution, and strict requirements for experimental conditions and storage environment to retain its activity. Because peroxidases contain iron porphyrin as their active sites, to artificially engineer metalloporphyrins into protein-like scaffold represents the major efforts on the works of peroxidase mimetics. Although they share common structural features of the active center of peroxidase, simple synthetically-modified hemin molecules, however, hardly show satisfactory activity and selectivity mainly due to the lack of the peptidic microenviorment that exists in the natural peroxidase. Since the structural amino acids or functional groups around the active site cause the special inclusion behavior between the enzyme and the substrate, β-Cyclodextrins (β-CDs), as one frequently used model system, act as an excellent enzyme model because of their fairly rigid and hydrophobic cavities that have appropriate size. Experimentally, β-CD-modified hemins have showed higher activity relative to free hemin. These promising results have inspired us to use supramolecular hydrogels to encapsulate hemin for the mimetic of peroxidase.

Although supramolecular hydrogels, formed by self-assembly of nanofibers of amphiphilic small molecules, have served as scaffolds for tissue engineering, medium for screening inhibitors of enzymes, matrix for biomineralization and biomaterials for wound healing, their application as the skeletons of artificial enzymes has yet to be explored. Similar to peptide chains chosen by nature for the backbone of active sites in enzymes, the self-assembled nanofibers of amino acids in the supramolecular hydrogels could offer the matrices of artificial enzymes. In other words, the supramolecular hydrogels systems serve two functions—as the skeletons of the artificial enzyme to aid the function of the active site (e.g., hemin) and as the immobilization carriers to facilitate the recovery of the catalyst in practical applications.

Experimental Methods

Formation of Gel II and Gel I: Addition of Fmoc-L-Phenylalanine (50 μmol), Fmoc-L-lysine (50 μmol), and sodium carbonate (100 μmol) to 1 ml water solution got a suspension mixture. After heating to 333 K, the suspension mixture turned to clear solution. Then 10 μmol hemin chloride powders were mixed and dissolved with the peptides solution. At last, a hydrogel composite with hemin (Gel II) was formed after about 10 minutes. Without adding hemin chloride, Gel I was formed. Substituting Fmoc-L-Phenylalanine (Hemin_{(Phe)}) with small molar amount Fmoc-L-Alanine, Fmoc-L-Valine, Fmoc-L-Leucine resulted in Hemin_{(Ala)}, Hemin_{(Val)}, and Hemin_{(Leu)}, respectively. To evaluate the effect of L-Histidine on hemin, L-Histidine (30, 20, 15, 10, 5 μmol) was mixed with hemin chloride (10 μmol) and then dissolved into Gel II. The encapsulated procedures of hemin within β-CD or a polyacrylamide were previously reported.

Activity assay: Using the oxidation of pyrogallol (10.0 mM) by H₂O₂ (40.0 mM) as a model reaction and fixing the total concentration of Hemin to 5 μM in the mixture, we examined the activity of Hemin by monitoring the absorbance (420 nm) of purpurogallin, the product of Hemin-catalyzed oxidation of pyrogallol. The catalytic reaction course of the artificial enzyme was measured by monitoring the increase in absorbance along with time change. The absorbance increase in the first minute was defined to be the initial rate. The Lineweaver-Burk plots constructed by the reaction initial rates at different pyrogallol concentrations were used to estimate their kinetic constant values. The other substrates, including o-phenyldiamine (product absorption at 450 nm) and o-aminophenol (product absorption at 430 nm), were employed under the same procedure of pyrogallol.

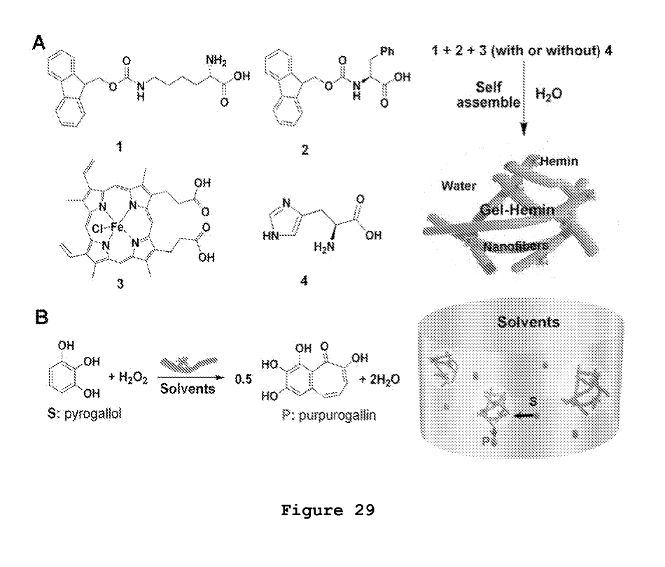

In the present invention, we mixed hemin chloride (3) into the supramolecular hydrogel formed by the self-assembly of two simple derivatives of amino acids (1 and 2) (see FIG. 29). We found that the activity of hemin in this type of artificial enzyme system was always higher than the activity of free hemin, hemin in β-CD, or hemin in polymeric hydrogels. This supramolecular hydrogel-based artificial enzyme shows the highest activity in toluene for an oxidation reaction, which reaches about 60% of the inherent activity of the most active peroxidase, HRP. These results are particularly interesting because it implies that tailoring the nanofibers via the control of the structure of hydrogelators provides adjustable microenvironment around active sites to optimize the performance of artificial enzymes. Additionally, it suggests a unique role of the self-assembled nanofibers in supramolecular hydrogels. Moreover, the supramolecular hydrogel acts as an effective carrier to minimize dimerization and auto-oxidization of single hemin in peroxidization reaction. Overall, the supramolecular hydrogel-based artificial enzyme offers a new opportunity to execute catalysis with high operational stability and reusability, which ultimately would benefit industrial biotransformation.

FIG. 29 illustrates the simple procedure to make the artificial peroxidase by using supramolecular hydrogels to encapsulate hemin. Equal moles of 1 and 2 and two equivalents of Na₂CO₃ were added to water to get a suspension, which turned into a clear solution at about 60° C. Then 3 was mixed and dissolved into the solution immediately. The subsequent cooling of the solution to room temperature afforded a supramolecular hydrogel containing hemin molecules (Gel II). Without adding 3, the same procedure gave the control (Gel I). For more completed comparison, we constructed the artificial peroxidase using β-CD or a polyacrylamide hydrogel to encapsulate hemin according to published reports.

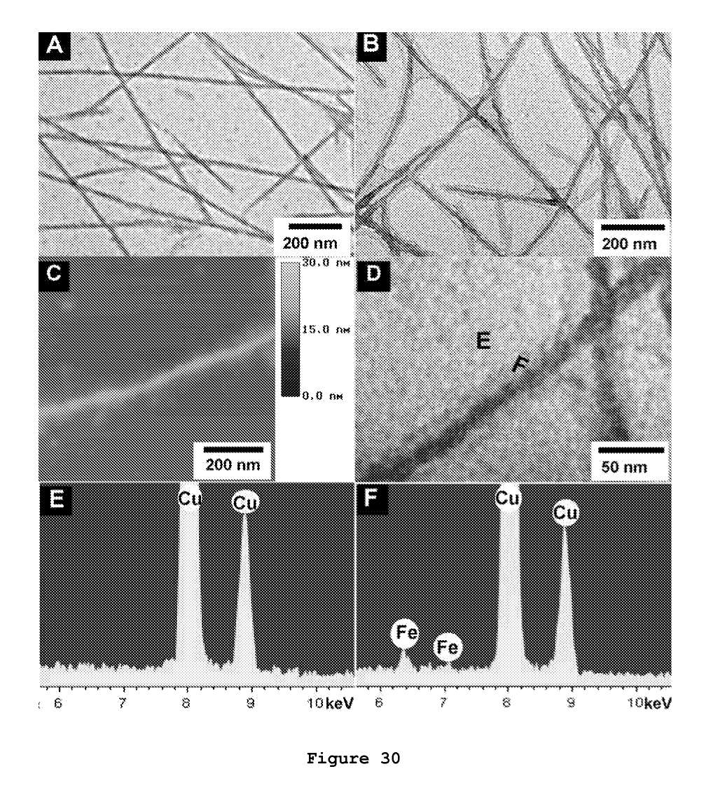

Both TEM and AFM images FIG. 30) reveal that Gel I and Gel II differ in morphorlogy. Gel I had large pore networks (200˜1000 nm in pore sizes) formed by the nanofibers (˜20 nm in width) of the self-assembly of compounds 1 and 2. TEM image of the nanofibers in Gel II, however, showed two distinct regions besides the reltively large pores, the dark part (fibers of ˜20 nm in diameter) and the gray part (surface layer of ˜6 nm thickness). The almost same size of dark part in Gel II as the nanofiber in Gel I indicated that the dark part mainly consisted of self-assembled nanofibers of 1 and 2. The gray part likely consisted of less ordered aggregates of 1 and 2. The AFM study of Gel II (FIG. 30C) also showed that a loose layer surrounds the dense nanofibers. The bright region in the AFM image corresponded to about 30 nm in height, agreeing with the TEM results. HRTEM of Gel II (FIG. 30D) revealed clearly the dark nanofibers being surrounded by the gray part, which agreed with the morphology observed by AFM. To probe the composition of gray part, different areas in HRTEM image of Gel II (areas E and F in FIG. 30D) were selected for EDX analysis. The blank area (FIG. 30B) had no Fe signal, while the area around nanofibers (FIG. 30F) had 5.06 atom % Fe. This comparison of EDX confirms the presence of hemin molecules in the outer layer of the nanofibers, a feature that would allow the substrate to approach hemin easily.

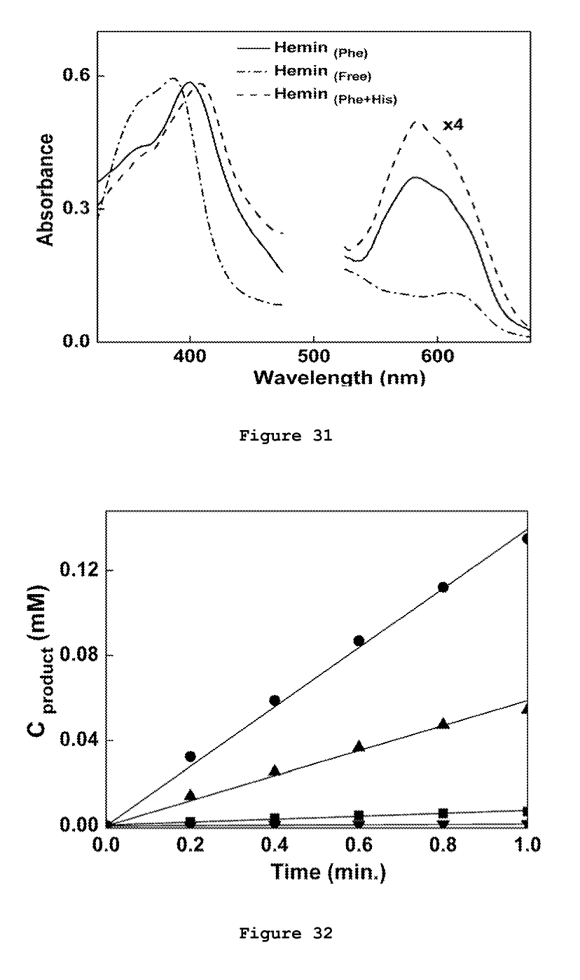

FIG. 31 shows the UV-Vis spectra of hemin in a buffer and the hydrogel matrices. The free hemin chloride in pH 7.4 buffer displays spectra with Soret peaks at 365 and 385 nm, indicating the presence of a mixture of both monomeric hemin hydroxide (haematin) and μ-oxo bihemin (oligomeric forms). A low intensity band around 610 nm also agreed with the Q band value of μ-oxo bihemin. Therefore, the predominant structures of hemin in pH 7.4 phosphate buffer were the hemin dimers connected by μ-oxo bridges in addition to some haematin. Gel II displayed a broad Soret band at 400 nm with a shoulder at 365 nm and a weak band at 585 nm. The Soret bands of the hemin inside hydrogel (at 400 nm, similar to hemin in aqueous micelle solutions and artificial proteins) agreed with the spectra of hemin chloride in DMSO and methanol, suggesting monomeric hemin chloride. The weak absorbance band at 585 nm should be ascribed to CT band (charge transfer transition from π electrons of porphyrin ring to d orbital of ferric ion). The weak shoulder 365 nm in the spectra of Gel II indicated small amount of the monomeric haematin. Overall, the nanofibers in the supramolecular hydrogel effectively reduced the dimerization of hemin via supramolecular interactions to localize monomeric hemin chloride within/around the nanofibers. With the addition of L-histidine, the spectrum of Gel II showed a red shift in Soret band from 400 nm to 406 nm. The change of Soret band was same as that of hemin in methanol after titrating by imidazole, indicating the formation of hemin-histidine complex through bond of Fe(III)-N.

Using the oxidation of pyrogallol as a model reaction and fixing the total concentration of Hemin to be 5 μM in the mixture, we obtained the catalytic rate of Hemin by monitoring the changes of the absorbance of purpurogallin, the product of Hemin-catalyzed oxidation of pyrogallol. As shown in FIG. 32, Gel I (i.e., the control) exhibited no catalytic activity, and Gel II (i.e., Hemin_{(Phe)}) exhibited higher activities than free Hemin (i.e., Hemin_{(Free)}) in the same buffer (10 mM, pH 7.4, phosphate), confirming the effectiveness of the artificial enzyme system derived from the hemin in the supramolecular hydrogel. The Lineweaver-Burk plots constructed by the reaction initial rates at different pyrogallol concentrations gave the activity of the artificial enzyme. The kinetics of the catalysis of artificial enzyme follows Michaelis-Menten equation, which indicated successful design and generation of an artificial peroxidase by encapsulating hemin into hydrogel. As shown in Table 3, the artificial enzyme had higher turnover number (k_{cat} value) than Hemin_{(Free)}, β-CD bound hemin (Hemin_{(β-CD)}), polyacrylamide hydrogel encapsulated hemin (Hemin_{(polymer)}). Two factors likely contribute to the high activity of Hemin_{(Phe)} in pH 7,4 buffer: (i) localization of hemin on the nanofibers preserves the catalytic species—monomeric hemin; (ii) the mesoporous structure in hydrogel facilitates the substrate across the hydrogel network to access the hemin in the hydrogel.

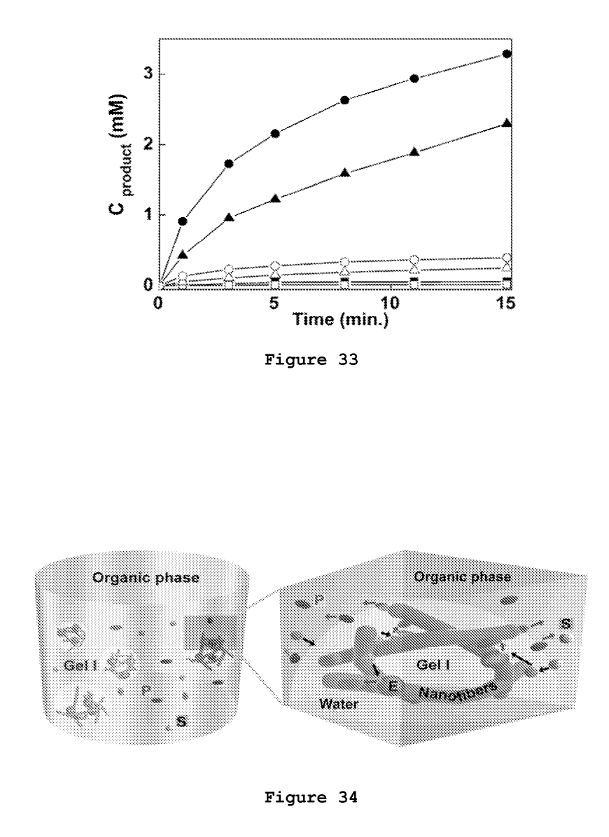

Another notable feature of the supramolecular hydrogel-based artificial enzyme is its high activity in an organic solvent. As shown in FIG. 33, the catalytic ability of Hemin_{(Phe)} in toluene was much higher than that of Hemin_{(Phe)} in water buffer. The kinetics data in Table 3 indicated that the k_{cat} value of Hemin_{(Phe)} in toluene (370 min⁻¹) was 18 times over that of Hemin_{(Phe)} in water buffer (19.9 min⁻¹). In addition, the value of Hemin_{(Phe)} in toluene was 136 times that of Hemin_{(Free)} in toluene (2.7 min⁻¹). The supramolecular hydrogel also greatly outperformed other hemin carriers such as β-CD or poly(acrylamide) hydrogel in all solvents tested. The k_{cat} value of Hemin_{(β-CD)} and Hemin_{(polymer)} in toluene were 6.1 min⁻¹ and 2.4 min⁻¹respectively, which were close to that of Hemin_{(Free)} in toluene. Therefore, the unique molecular arrangement of amino acids in the supramolecular hydrogels—nanofibers—likely results in high activity of the artificial enzyme in toluene. Additionally, the aqueous environment of the hydrogel promotes the hydrophilic substrate (i.e., pyrogallol) across the microinterface of H₂O/toluene to enter the hydrogel, and the amphiphilic hydrogelators and/or the mesoporous structure of the self-assembled nanofiber in Gel II may also assist the substrates to approach hemin and the products to leave the active site.

TABLE 3
Kinetic Constants of Various Systems^{a}

| | k_{cat} in buffer (min⁻¹) | k_{cat} in toluene (min⁻¹) |
|---|---|---|
| Hemin_{(Phe)} | 19.9 | 370.7 |
| Hemin_{(Phe + His)} | 49.7 | 1045.3 |
| Hemin_{(Free)} | 2.4 | 2.7 |
| Hemin_{(β-CD)} | 4.8 | 6.1 |
| Hemin_{(β-CD + His)} | 5.6 | 7.8 |
| Hemin_{(polymer)} | 7.6 | 2.4 |
| Hemin_{(polymer + His)} | 9.2 | 2.8 |
| Met-Hb | 37.4 | 1.0 |
| HRP | 1740.0 | 1.8 |

^{a}The background has been substracted.

FIG. 33 also lists the catalytic courses of the oxidation of pyrogallol catalyzed by Hemin_{(Phe+His)} in water buffer and toluene, respectively, verifying that L-histidine significantly increased the performance of the artificial enzyme. As the kinetic parameters listed in Table 3, the addition of histidine dramatically enhanced the activity of hemin in the artificial enzyme to about 2.5 times in buffer and 2.8 times in toluene, indicating that the coordination of histidine to the Fe(III) center in the hemin resulted in the activity changes in the artificial enzymes. As shown in FIG. 31, the Soret band value of artificial enzyme was similar to that of methemoglobin (408 nm) and peroxidase (405 nm), which both have the proximal histidine ligand binding with the iron ion in heme. It is well known that the proximal histidine in preoxidase makes important contribution for the activity. Therefore, the peroxidase-like hemin-histidine complex in the artificial enzymes should be the major reason for the higher activity relative to the artificial enzymes without L-histidine. This hypothesis agrees with that the activity enhancement is the same in different solvents and different carriers (Table 3).

The supramolecular hydrogel-immobilized hemin also exhibited high stability and excellent reusability, which is particularly relevant for industrial applications. FIG. 33 shows the 15 minutes courses of the oxidation of pyrogallol catalyzed by various hemin in water buffer and toluene, respectively. The hemin in the hydrogels remained catalytic ability after 15 min reaction in toluene and in water, while the native hemin lost most catalytic ability after 5 min. To test its reusability, we used fresh and recovered artificial enzyme (Hemin_{(Phe)}) to catalyze the peroxidization of pyrogallol in toluene. The result shows that the amount of product in third run reached 82 percent of that in first run. Two plausible reasons can explain the high stability of the artificial enzyme in toluene system: Firstly, the supramolecular hydrogel provides an aqueous microenvironment to protect hemin from deactivating by the organic solvent. Secondly, the separation of hemin molecules in nanofiber networks avoids auto-oxidized inactivation of hemin.

To verify the generality of the supramolecular hydrogels as the skeleton and carrier of artificial enzymes, we used other Fmoc amino acids to assemble various hydrogels as the hemin carriers and evaluated the activity of hemin in these hydrogels. Hemin_{(Ala)}, Hemin_{(Val)}, and Hemin_{(Leu)} have similar activity as that of Hemin_{(Phe)}, in either buffer or toluene. Various substrates also were employed to evaluate the catalytic ability of artificial enzyme in toluene. The result shows that the activity of Hemin_{(Phe+His)} in toluene can arrive at 20.2 and 4.2 percent of that of HRP in water with o-phenyldiamine and o-aminophenol as substrate, respectively.

In summary, we demonstrate that nanofibers in the supramolecular hydrogels, formed by simple derivative of amino acids, act as the skeletons of the artificial enzyme and the hydrogels also serve as immobilized carriers to enhance the catalytic activity of hemin chloride for peroxidation. The artificial enzyme not only mimics the function of peroxidases in water, but achieves relative high activity. The highest activity of artificial enzyme in toluene even arrive at about 60% that of the most active peroxidase HRP in water. The main advantage of supramolecular hydrogels in the artificial enzyme is the novel microenvironment that protects the hemin monomer by overcoming dimerization and auto-oxidation and to facilitate catalytic reaction by providing mesoporous diffusion channels, which possess unique flexibility to allow the transport of a large range of substrates. Moreover, the principle illustrated in this example may allow synthesis of artificial enzymes by tailoring molecular hydrogelator to mimic the functions of natural enzymes in media such as organic solvents. This general strategy can be applied to a variety of artificial systems and gels. Due to the distinct technological advantages, enzymatic catalysis in organic solvents attracts many attentions, which is unexplored for previous artificial enzymes. In the present invention, the artificial enzyme in supramolecular hydrogels can execute biocatalysis in toluene by the designed microheterogeneous system.

### EXAMPLE 15

### Supramolecular Hydrogels-Encapsulated Enzyme for Biotransformation

Enzymatic catalysis in organic solvents offers distinct technological advantages (e.g., enhanced stability of enzymes, easy recovery of products, and novel biosynthesis) for a variety of applications and has already led to some successful commercial processes. Enzymes, however, often display drastically lower activity in organic solvents than in water. Klibanov et al. have elucidated that the water layer on the molecular surface of enzymes determines their activity in organic media and have suggested three major causes of the low activity—unfavorable substrate desolvation, suboptimal ph, and reduced conformational mobility. Among several known approaches to remedy these problems, it is quite effective to immobilize enzymes within an aqueous microenvironment in the organic solvent. For example, enzymes bound inside polymer hydrogels or organic plastics show enhanced activity and stability relative to native enzymes in organic media, and the enzymes within the water phase of reverse micelles exhibit near or even higher activity in organic media than that in water. These promising results lead us to develop molecular hydrogels as new materials to immobilize enzymes for catalysis in organic media.

It is rather simple to make a molecular hydrogel for confining an enzmye. Mixing sodium carbonate (20 mg), Fmoc-L-lysine (36 mg), and Fmoc-L-phenylalanine (38 mg) into 0.9 mL water gave a suspension which turned into a clear solution upon heating to about 333 K. The addition of 0.1 mL hemoglobin (Hb, 40 mg) into the solution at 308˜313 K and the subsequent cooling to room temperature resulted in Gel I. A similar procedure allows the immobilization of other enzymes (e.g., horseradish peroxidase (50 U), laccase (3.6 U), or alpha-chymotrypsin (100 U)). Without the addition of enzymes, the same process produces Gel II as a control. A crosslinked poly(acrylamide) hydrogel containing Hb serves as another control (Gel III). The rheological test confirms the elastic nature of Gel I and Gel II. The dynamic storage modulus of Gel I was ten times lower than that of Gel II, indicating that crosslinks in Gel I existed at lower density than that in Gel II and suggesting that the interaction of Hb with the nanofibers decreased the density of the crosslink. TEM images revealed that Hb molecules aggregated and shorten the nanofibers (˜16 nm in diameters) made of the hydrogelators, thus reducing the density of crosslink in Gel I. Both AFM and TEM images indicated that Hb molecules mainly locate at the crosslink sites of the nanofibers, which agreeed with the Theological data. Little release of Hb from Gel I into solvents also confirmed that the non-covalent interaction between the enzyme and the nanofibers was strong enough to ensure the immobilization.

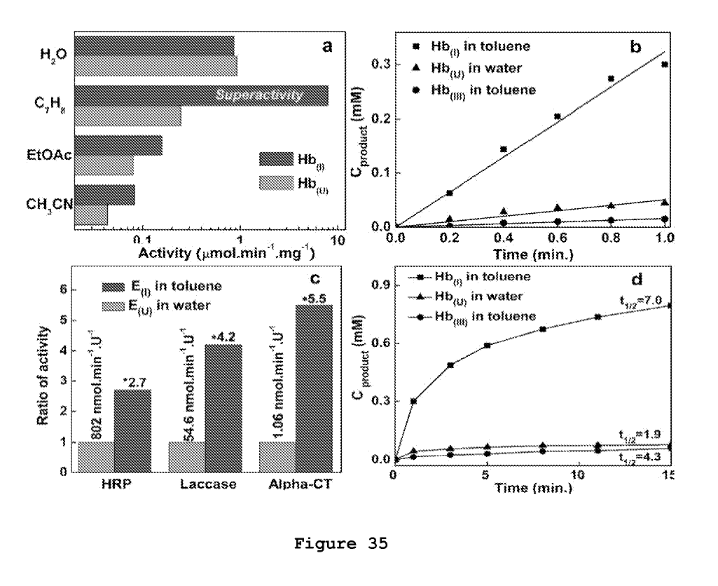

Since enzymatic catalyses in organic solvents have mainly used hydrolytic enzymes, and oxidoreductases were almost unexplored, we first examined the activity of Hb (as a substitute of peroxidase, and in the form of HbFe(III)) in its unconfined form and inside hydrogels for catalyzing oxidation in different solvents at room temperature. Using oxidation of pyrogallol by H₂O₂ as the model reaction, we examined the activity of Hb by monitoring the concentration of purpurogallin. The control, Gel II, exhibits no activity. As shown in FIG. 35A, Gel I-bound Hb (Hb(I)) exhibited almost the same activities as the unconfined Hb (Hb(U)) did in water, suggesting that the structures of Hb(I) and Hb(U) differ little, which agrees with the observations that the UV/Vis and CD spectra of Hb(U), Hb(I), and Hb(III) displayed little or no structural change.

Hb(I) exhibited higher activities than Hb(U) did in the same organic media tested, confirming the protective effect of the aqueous microenvironment provided by Gel I. The activities of both Hb(I) and Hb(U) increased with the decrease of the polarity of the organic solvent (from acetonitrile to toluene), which agrees with the established trend of the activity of an enzyme in an organic solvent. The Lineweaver-Burk plots constructed by the reaction initial rates at different pyrogallol concentrations were used to estimate their kinetic constant values. The activity of Hb(I) in toluene (7.98 μmol.min⁻¹.mg⁻¹) was eight times more active than Hb(U) in bulk water (0.92 μmol.min⁻¹.mg⁻¹). According to FIG. 35B, the initial rate of Hb(I) at 10 mM pyrogallol concentration was much larger than that of Hb(U) and Hb(III). These results represent the first observation of the superactivity of an enzyme confined in a medium other than reverse micelles.

Other molecular hydrogel-immobilized enzymes also displayed superactivity in organic media (FIG. 35C), indicating the generality of the superactivity conferred by the molecular hydrogel. We suggest that it is likely that several factors contribute to the superactivity of Hb(I): (i) Hydrophilicity promotes the substrate (i.e., pyrogallol) across the microinterface to enter the hydrogel, similar to the case of reversed micelles; (ii) Amphiphilic character and/or the molecular superstructure of the self-assembled nanofiber in Gel I may assist the substrates to approach Hb and the products to leave Hb. This assumption agrees with the much lower activity of Hb(III) (i.e., Hb immobilized by a randomly-crosslinked poly(acrylamide) hydrogel) than that of Hb(I); (iii) The large pore sizes of the nanofibrous networks in Gel I (TEM and AFM confirm that 0.2-2 μm and 5-6 nm pores in Gel I and Gel III, respectively) facilitate the mass transport in Gel I.

Molecular hydrogels also significantly improve the stability of the enzymes. As shown in FIG. 35D, Hb(I) had improved stability in toluene compared with that of Hb(U) in water. The quantitative analysis of their reaction course shows the highest stability of Hb(I) in toluene, as indicated by the half lives (t½) of Hb. To evaluate the potential industrial application of the system, we chose 2-aminophenol (3) as another substrate for Hb(I) catalyzed oxidization in toluene because the oxidative product of 3 is 2-amino-3H-phenoxazin-3-one (4, a useful antibiotic called questiomycin A). Although the initial rate of Hb(I) in toluene was slightly lower than that of Hb(U) in water, indicating that the superactivity was also substrate dependent, the molecular hydrogel significantly improved the stability of Hb(I) in toluene (t½=27.8 minutes) and led to the additional production of 4 in an hour. We also employed this reaction to test its stability as a recovered catalyst. The first run achieved 98% conversion of 3, and the second and third runs of reused Hb(I) obtained 97.0% and 95.0%, respectively. Almost the same conversion of the first and third runs indicates that the Hb(I) in toluene can be reused without losing activity. Two plausible reasons can explain the observed high stabilities: Firstly, the molecular hydrogel provides an aqueous microenvironment that protects the enzyme from deactivation by the organic solvent. Secondly, the relative large pore size and amphiphilic nature of the molecular hydrogel facilitate the transport of the product back to the organic phase, thus reducing inhibition of the catalyst. The second reason also explains the short t½ of Hb(III) in toluene (t½=14.1 minutes) on account of the trapping of the product in the hydrogel due to small pore sizes.

In summary, we have demonstrated that molecular hydrogels provide a unique aqueous microenvironment in which to carry out enzymatic reactions in an organic solvent. Our observation also suggests that molecular hydrogel may lead to a general strategy, which combines the reusability of polymer hydrogels and the high activity of the reversed micelles, to perform enzyme catalyzed transformation in organic media. The self-assembled nanofibers in molecular hydrogels also offer a new opportunity to engineer the immobilization medium in organic solvents for superactivity, high operational stability, and reusability of enzymes, which ultimately will benefit industrial biotransformation. Moreover, the principle illustrated herein may allow immobilization of catalysts in organogels to carry out reactions in water. This general strategy can be applied to a variety of catalysts and gels.

### REFERENCES

Appella et al., Beta-peptide foldamers: Robust Helix formation in a new family of beta-amino acid oligomers. Journal of the American Chemical Society 118:13071-13072 (1996).
Ashiku et al., Mater. Sci. Forum 250:129-150 (1997).
Burch et al., N-(Fluorenyl-9-Methoxycarbonyl)Amino-Acids, a Class of Antiinflammatory Agents with a Different Mechanism of Action. Proceedings of the National Academy of Sciences of the United States of America 88:355-359 (1991).
Estroff and Hamilton, Chem. Rev. 104:1201 (2004).
Giannis, Peptidomimetics For Receptor Ligands Discovery, Development, And Medical Perspectives. Angew. Chem. Intl. Ed. 32:1244-1267 (1993).
Hirobe, Development 102:567 (1988).
Hook et al., The proteolytic stability of ‘designed’ beta-peptides containing alpha-peptide-bond mimics and of mixed alpha,beta-peptides: Application to the construction of MHC-binding peptides. Chemistry & Biodiversity 2:591-632 (2005).
Hu and Messersmith, Rational Design of Transglutaminase Substrate Peptides for Rapid Enzymatic Formation of Hydrogels. J. Am. Chem. Soc. 125:14298-14299 (2003).
Lee et al., Adv. Mater. 15:1828-1832 (2003).
Martinek and Fulop, Side-chain control of beta-peptide secondary structures—Design principles. European Journal of Biochemistry 270:3657-3666 (2003).
Porter et al., Antibiotics—Non-haemolytic beta-amino-acid oligomers. Nature 404:565-565 (2000).
Seebach and Matthews, Beta-peptides: a surprise at every turn. Chemical Communications (21), 2015-2022 (1997).
Seebach et al., Beta(2)- and beta(3)-peptides with proteinaceous side chains: Synthesis and solution structures of constitutional isomers, a novel helical secondary structure and the influence of solvation and hydrophobic interactions on folding. Helvetica Chimica Acta. 81:932-982 (1998).
Tabata et al., Pure Appl. Chem. 70:1277-1282 (1998).

What is claimed is:

1. A supramolecular hydrogel having a three-dimensional, self-assembling, network structure comprising non-polymeric, functional molecules and a liquid medium, wherein the functional molecules are noncovalently crosslinked.

2. The hydrogel of claim 1, wherein the noncovalent crosslinking is effectuated by an interaction selected from the group consisting of ligand-receptor interaction, hydrogen bonding, hydrophobic interaction, and ionic interaction.

3. The hydrogel of claim 1, wherein the liquid medium is water or physiological saline.

4. The hydrogel of claim 1, wherein the non-polymeric functional molecules are selected from the group consisting of anti-inflammatory molecules, antibiotics, metal chelators, anticancer agents, small peptides, and surface-modified magnetic nanoparticles.

5. The hydrogel of claim 2, wherein the ligand is vancomycin and the receptor is a D-Ala-D-Ala derivative.

6. The hydrogel of claim 4, wherein the non-polymeric functional molecules comprise a naphthalene group.

7. The hydrogel of claim 4, wherein the small peptides are selected from the group comprising single amino acids, dipeptides, tripeptides, tetrapeptides, β-amino acids, pentapeptides, and derivatives thereof, wherein the molecular weight of the small peptides are less than 3.0 KD.

8. The hydrogel of claim 4, wherein the anti-inflammatory molecules are selected from the group consisting of N-(Fluorenyl-9-methoxycarbonyl)-L-Leucine and N-(Fluorenyl-9-methoxycarbonyl)-L-Lysine.

9. The hydrogel of claim 4, wherein the antibiotics are selected from the group consisting of vancomycin, penicillin, amoxicillin, cephalosporin, oxacillin, nafcillin, clindamycin, erythromycin, ciprofloxacin, rifampin, amphotericin, and sulfamethoxaole.

10. The hydrogel of claim 4, wherein the metal chelators are chelating agents for radioactive isotopes.

11. The hydrogel of claim 7, wherein the small peptide is selected from the group comprising naphthalene-containing amino acids and dipeptides, and their derivatives thereof, Nap-D-Phe-D-Phe, Nap-s-β³-HPhg-s-β³-HPhg and Nap-L-fPhe-L-fPhe.

12. The hydrogel of claim 7, wherein the β-amino acids or their derivatives are selected from the group comprising β³-alanine, β³-phenylalanine and β³-HPhg.

13. The hydrogel of claim 12, wherein the hydrogel comprising β-amino acids has enhanced biostability compared to hydrogel that does not contain β-amino acids.

14. A method of treating wounds, comprising the step of administering the hydrogel of claim 1 to an external or internal wound of a subject in need thereof.

15. The method of claim 14, wherein the hydrogel comprises non-polymeric functional molecules having a naphthalene group.

16. The method of claim 14, wherein the non-polymeric functional molecules comprise glucosamine.

17. A method of making a supramolecular hydrogel, comprising the use of a precursor of hydrogelators that are hydrolyzed by a hydrolyase under proper conditions, thereby generating hydrogelators that form the hydrogel.

18. The method of claim 17, wherein the hydrolyase is selected from the group consisting of alkaline phosphatase, acid phosphatase, esterase, amidase, and peptidase.

19. The method of claim 17, wherein the hydrogelators are selected from the group comprising FMoc-substituted amino acids and their derivatives thereof.

20. A method of screening a candidate compound for its ability to inhibit an enzymatic reaction, comprising the steps of:
a. providing a precursor which transforms into a hydrogelator in the presence of an enzyme;
b. contacting the precursor with the enzyme; and
c. determining the formation of hydrogel by the hydrogelator, wherein inhibition of hydrogel formation in the presence of the candidate compound indicates that the candidate compound is an enzyme inhibitor.

21. The method of claim 20, wherein the precursor is selected from the group comprising FMoc-substituted amino acids and their derivatives thereof.

22. The method of claim 20, wherein the enzyme is from an organism selected from the group consisting of bacteria, viruses, and parasites.

23. A method of screening a test sample for the presence of an enzyme, comprising the steps of:
a. providing a precursor which transforms into a hydrogelator in the presence of the enzyme;
b. contacting the precursor with the test sample; and
c. determining the formation of hydrogel by the hydrogelator, wherein hydrogel formation in the presence of the test sample indicates that the test sample contains the enzyme.

24. The method of claim 23, wherein the enzyme is from an organism selected from the group consisting of bacteria, viruses, and parasites.

25. A method of delivering a therapeutic agent, comprising the step of using the hydrogel of claim 1 as a carrier for the therapeutic agent.

26. The method of claim 25, wherein the hydrogel comprises β-amino acids.

27. A method of conducting an enzymatic reaction, comprising the step of enclosing an active site of an enzyme in the hydrogel of claim 1.

28. The method of claim 27, wherein the enzymatic reaction takes place in water or organic solvent.

29. A method of culturing cells, comprising the use of the hydrogel of claim 1 as a three-dimensional matrix for cell growth.


SEQUENCE LISTING (6 sequences)

SEQ ID NO: 1 - protein, 5 aa, Artificial Sequence
  pentapeptide
GAGAS

SEQ ID NO: 2 - protein, 5 aa, Artificial Sequence
  pentapeptide
GVGVP

SEQ ID NO: 3 - protein, 5 aa, Artificial Sequence
  pentapeptide
VPGVG

SEQ ID NO: 4 - protein, 5 aa, Artificial Sequence
  pentapeptide
VTEEI

SEQ ID NO: 5 - protein, 5 aa, Artificial Sequence
  pentapeptide
VYGGG

SEQ ID NO: 6 - protein, 5 aa, Artificial Sequence
  pentapeptide
YGFGG